SEQUENCE LISTING (22 sequences)

SEQ ID NO: 1 - DNA, 20 bp, Artificial Sequence
  Description of Artificial Sequence Synthetic oligonucleotide
aacaactatcgttaccacag

SEQ ID NO: 2 - DNA, 20 bp, Artificial Sequence
  Description of Artificial Sequence Synthetic oligonucleotide
agacgggcacgaggttccct

SEQ ID NO: 3 - DNA, 20 bp, Artificial Sequence
  Description of Artificial Sequence Synthetic oligonucleotide
tcacgcacaagaaacgtcca

SEQ ID NO: 4 - DNA, 20 bp, Artificial Sequence
  Description of Artificial Sequence Synthetic oligonucleotide
accctgatgggacgtacact

SEQ ID NO: 5 - DNA, 20 bp, Artificial Sequence
  Description of Artificial Sequence Synthetic oligonucleotide
aggcacagtagaagccgtat

SEQ ID NO: 6 - DNA, 20 bp, Artificial Sequence
  Description of Artificial Sequence Synthetic oligonucleotide
gacggctgaggagcggaaga

SEQ ID NO: 7 - DNA, 20 bp, Artificial Sequence
  Description of Artificial Sequence Synthetic oligonucleotide
tgtggaggtgagcaatcccc

SEQ ID NO: 8 - DNA, 20 bp, Artificial Sequence
  Description of Artificial Sequence Synthetic oligonucleotide
aggccacatagtgctgcaca

SEQ ID NO: 9 - DNA, 20 bp, Artificial Sequence
  Description of Artificial Sequence Synthetic oligonucleotide
tgtacagcagtggctggtgg

SEQ ID NO: 10 - DNA, 20 bp, Artificial Sequence
  Description of Artificial Sequence Synthetic oligonucleotide
ccccttcggtcaccacgagc

SEQ ID NO: 11 - DNA, 20 bp, Artificial Sequence
  Description of Artificial Sequence Synthetic oligonucleotide
ctcctcggtgtacatcacgg

SEQ ID NO: 12 - DNA, 20 bp, Artificial Sequence
  Description of Artificial Sequence Synthetic oligonucleotide
agtagttggtgacgttctgc

SEQ ID NO: 13 - DNA, 20 bp, Artificial Sequence
  Description of Artificial Sequence Synthetic oligonucleotide
cgctacacggtgctgagcgt

SEQ ID NO: 14 - DNA, 20 bp, Artificial Sequence
  Description of Artificial Sequence Synthetic oligonucleotide
gagttccgtgaccgtgcaag

SEQ ID NO: 15 - DNA, 20 bp, Artificial Sequence
  Description of Artificial Sequence Synthetic oligonucleotide
tgagttcccaacttcattag

SEQ ID NO: 16 - DNA, 20 bp, Artificial Sequence
  Description of Artificial Sequence Synthetic oligonucleotide
tgagaagcgacagccagtga

SEQ ID NO: 17 - DNA, 20 bp, Artificial Sequence
  Description of Artificial Sequence Synthetic oligonucleotide
ggccagactggcaccaacgg

SEQ ID NO: 18 - DNA, 20 bp, Artificial Sequence
  Description of Artificial Sequence Synthetic oligonucleotide
ctactgaagtatacgtaaag

SEQ ID NO: 19 - DNA, 20 bp, Artificial Sequence
  Description of Artificial Sequence Synthetic oligonucleotide
catctatcggaacacttcat

SEQ ID NO: 20 - DNA, 20 bp, Artificial Sequence
  Description of Artificial Sequence Synthetic oligonucleotide
cgaaaggaaccacgctggtc

SEQ ID NO: 21 - DNA, 20 bp, Artificial Sequence
  Description of Artificial Sequence Synthetic oligonucleotide
atttactgtcacggttccca

SEQ ID NO: 22 - DNA, 20 bp, Artificial Sequence
  Description of Artificial Sequence Synthetic oligonucleotide
ccccatagatgattatgcat

(19) United States
(12) Patent Application Publication
(10) Pub. No.: US 2022/0389383 A1
(43) Pub. Date: Dec. 8, 2022

(54) LARGE-SCALE COMBINED CAR TRANSDUCTION AND CRISPR GENE EDITING OF NK CELLS

(71) Applicant: BOARD OF REGENTS, THE UNIVERSITY OF TEXAS SYSTEM, Austin, TX (US)

(72) Inventors: Katy REZVANI, Houston, TX (US); Rafet BASAR, Houston, TX (US); May DAHER, Houston, TX (US); David MARIN COSTA, Houston, TX (US)

(21) Appl. No.: 17/755,887

(22) PCT Filed: Nov. 25, 2020
(86) PCT No.: PCT/US2020/062372

§ 371 Date: May 11, 2022

Related U.S. Application Data
(60) Provisional application No. 62/941,134, filed on Nov. 27, 2019.

(51) Int. Cl. C12N 5/0783; A61K 35/17; A61P 35/00; C12N 15/113

(52) U.S. Cl. CPC C12N 5/0646; A61K 35/17; A61P 35/00; C12N 15/1138; C12N 2310/20; C12N 2501/2302

(57) ABSTRACT

Embodiments of the disclosure encompass methods and compositions for producing engineered natural killer (NK) cells. The disclosure concerns large-scale processes for producing NK cells that are engineered to have disruption of expression of one or more genes, such as using CRISPR, and also engineered express at least one heterologous antigen receptor. Specific embodiments include particular parameters for the process.

This application claims priority to U.S. Provisional Patent Application Ser. No. 62/941,134, filed Nov. 27, 2019, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Nov. 12, 2020, is named UTSC_P1199WO_SL.txt and is 5,505 bytes in size.

### TECHNICAL FIELD

The present disclosure relates generally at least to the fields of immunology, cell biology, molecular biology, and medicine.

### BACKGROUND

Cellular immunotherapy holds much promise for the treatment of cancer. However, most immunotherapeutic approaches when applied alone are of limited value against the majority of malignancies, including solid tumors. Reasons for this limited success include (1) reduced expression of tumor antigens on the surface of tumor cells, which reduces their detection by the immune system; (2) the expression of ligands for inhibitory receptors, such as PD1, NKG2A, TIGIT; (3) upregulation of cellular checkpoints, such as CISH that induce immune cell inactivation; and (4) the induction of cells (e.g., regulatory T cells or myeloid-derived suppressor cells) in the microenvironment that release substances such as transforming growth factor-β (TGFβ) and adenosine that suppress the immune response and promote tumor cell proliferation and survival. Thus, there is an unmet need for improved methods of cellular immunotherapy that at least in some embodiments address these challenges.

### SUMMARY

Embodiments of the disclosure encompass novel, methods and compositions for large-scale approaches (including GMP grade) for the combined CAR transduction and CRISPR gene editing of natural killer (NK) cells. The disclosure provides for engineered NK cells that express one or more heterologous antigen receptors and that also have been gene edited to have disruption of one or more endogenous genes in the NK cells. In specific embodiments, the heterologous antigen receptors are engineered antigen receptors that have been produced by the hand of man, for example to be fusion proteins comprising components from different sources. In some embodiments, CAR-transduced NK cells are engineered to have disruption of expression of one or more endogenous genes in the NK cells, and in other embodiments NK cells having disruption of expression of one or more endogenous genes in the NK cells are transduced or transfected (e.g., modified) to express one or more heterologous antigen receptors. Particular embodiments provide for large-scale CRISPR/Cas9-mediated engineering strategies of primary CAR-transduced NK cells.

In certain embodiments, the process utilizes specific parameters that allow the process to be large-scale, including for engineering up to 1×10⁹ or more cells. In particular embodiments, the process utilizes successive steps including, in no particular order, optionally one or more expansion steps, optionally one or more depletion steps to remove cells positive for one or more markers, modification of the NK cells to have gene editing of one or more endogenous genes, and modification of the NK cells to express a heterologous antigen receptor. Any expansion step in any method encompassed herein may be optional. In specific cases, the process utilizes successive steps including one or multiple expansion steps, one or more depletion steps to remove cells positive for CD3, CD14, and/or CD19, modification of the cells to allow the NK cells to express one or more heterologous antigen receptors, and modification of the cells to lack expression, or have reduced expression, of one or more genes endogenous to the NK cells. In specific cases, 1, 2, 3, 4, 5, 6, 7, 8, 9, 10, or more endogenous genes are knocked out or knocked down using suitable mean, such as CRISPR and guide RNAs, for multiple genes. The process also encompasses specific durations of time for and/or between particular steps of the process.

The produced cells may be used for any purpose, including for the treatment of a medical condition such as cancer, infectious disease, and/or immune-related disorders. The produced cells may be stored, such as for an off-the-shelf purpose that upon their use may or may not be further modified, such as to customize the cells to an individual in need thereof. In some cases, no further modification is required, because the CAR expressed by the NK cells targets an antigen for an individual in need of targeting of the antigen (such as the antigen being a cancer (including tumor) antigen for a cancer of the individual). In other cases, the NK cells are stored before they have been modified to express a heterologous antigen receptor, and following storage and prior to use they are modified to express a CAR that targets an antigen for an individual in need of targeting of the antigen. The produced cells may be cryopreserved.

Embodiments of the disclosure comprise in vitro methods of producing engineered NK cells, comprising the steps of:

(a) expanding for a first time period NK cells (that may be derived from cord blood, peripheral blood, bone marrow, hematopoietic stem cells, induced pluripotent stem cells, cell lines, or a mixture thereof) with antigen presenting cells and an effective amount of one or more suitable cytokines, such as interleukin (IL)-2, IL-15, IL-21, and/or IL-18, to produce expanded NK cells;

one of (b1) and (c1), or (b2) and (c2) as follows:

(b1) transducing or transfecting the expanded NK cells of (a) with a vector encoding a heterologous antigen receptor to produce modified NK cells;

(c1) after a second time period, gene editing of the NK cells, such as delivering to the modified NK cells an effective amount of Cas9 and one or more guide RNAs to disrupt expression of one or more genes in the NK cells, thereby producing gene edited modified NK cells; or

(b2) gene editing of the expanded NK cells, such as delivering to the expanded NK cells of (a) an effective amount of Cas9 and one or more guide RNAs to disrupt expression of one or more genes in the NK cells, thereby producing gene edited NK cells;

(c2) after a second time period, transducing or transfecting the gene edited NK cells with a vector encoding a heterologous antigen receptor to produce gene edited modified NK cells; and

(d) optionally expanding for a third time period the gene edited modified NK cells of (c1) or (c2) in the presence of antigen presenting cells and an effective amount of IL-2. In specific cases, the gene edited modified NK cells are produced from the steps of (b1) and (c1) or from the steps of (b2) and (c2). In at least some cases, the first time period is between about 1-7 days, including 1-4 days or 5-7 days, as examples. The second time period may be about 1-4 days, including 1, 2, 3, or days. The third time period may be at least about 7 days, including between about 1-2 weeks.

In particular cases for the method, the steps of (c1) and (b2) are each further defined as two or more delivering steps. In such cases, a first delivering step may comprise delivering guide RNAs that target one or more genes and a second delivering step may comprise delivering guide RNAs that target one or more genes that are different from the one or more genes in the first delivering step. In specific examples, the duration between the first and second delivering steps is at least about 1, 2, 3, or 4 days or more and/or the duration between the first and second delivering steps is about 1-4, 1-3, 1-2, 2-4, 2-3, 3-4, or 1, 2, 3, 4, or more days.

In particular embodiments, one or more compositions that are delivered to the NK cells are delivered by electroporation. In cases wherein cells are electroporated, an electroporation may use between about 200,000 cells to 1×10⁹ NK cells. An electroporation may use between about 200,000 and 2,000,000 cells. An electroporation may use between about 1,000,000 to 1×10⁹ NK cells.

In particular embodiments, a method may further comprise a step of depleting NK cells of CD3-positive, CD14-positive, and/or CD19-positive cells, such as prior to (a). Any of the methods of the disclosure may further comprise the step of depleting NK cells of CD3-positive, CD14-positive, and/or CD19-positive cells prior to step (b1) or (b2). In some cases, in (a) and/or (d) the ratio of NK cells to antigen presenting cells is 1:1, 1:1.5, 1:2, or 1:3. The concentration of the guide RNA in the electroporation step is 3, 4, or 5 μM, in at least specific cases. The concentration of the Cas9 nuclease in the electroporation step may be about 4.0, 4.1, 4.2, 4.3, 4.4, 4.5, 4.6, 4.7, 4.8, 4.9, or 5μM, as examples. The concentration of IL-2 is between about 100 units/ml to about 300 units/ml, in specific cases. The concentration of IL-2 may be 100, 125, 150, 175, 200, 225, 250, 275, or 300 units/ml, in some cases.

In particular embodiments, the heterologous antigen receptor is a chimeric antigen receptor or a T cell receptor, although a single NK cell may expess one or more of them both. The heterologous antigen receptor may target a tumor associated antigen. In specific cases, the heterologous antigen receptor targets an antigen selected from the group consisting of CD19, CD319 (CS1), ROR1, CD20, CD70, B7H3, HLA-G, CD38, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD5, CD123, CD23, CD30, CD56, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, and a combination thereof.

In particular embodiments, the gene that has disruption of expression in the NK cells is any gene, but in specific cases it is an inhibitory gene, such as an inhibitory gene selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, CD39 and a combination thereof. In specific cases, the gene is an inhibitory gene.

In any of the methods herein, following the step of (d) the cells may or may not be analyzed, including the cells being analyzed by gene editing efficiency (such as knockout (KO) efficiency), functional assays, cytoxicity assays, and/or in vivo activity. In specific embodiments, the cells are analyzed for expression status, cytokine production, degranulation, cytotoxicity, anti-tumor activity, or a combination thereof. Analysis methods may include PCR, flow cytometry, mass cytometry, RNA sequencing, Incucyte killing, BLI imaging, chromium release assay, Annexin V apoptosis assay, or a combination thereof. Following the step of (d), the cells may be stored, such as cryopreserved. Following (d), an effective amount of the cells may be delivered to an individual in need thereof, such as one that has cancer, an infectious disease, and/or an immune-related disorder.

Embodiments of the disclosure include populations of NK cells produced by any method encompassed herein. Compositions comprising the population of cells of the disclosure are contemplated, including when the population is in a pharmaceutically acceptable carrier.

Embodiments of the disclosure include methods of treating an individual for a medical condition, comprising the step of administering to the individual a therapeutically effective amount of NK cells produced by any method of the disclosure.

Other objects, features and advantages of the present disclosure will become apparent from the following detailed description. It should be understood, however, that the detailed description and the specific examples, while indicating particular embodiments of the disclosure, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic diagram of protocol combining CAR transduction and CRISPR Cas9 gene editing of primary cord blood derived NK cells. CB NK cells are first expanded with feeder cells (APCs) and IL-2, on day 5 of culture NK cells are transduced with the CAR construct via retroviral vector, on day 7 this is followed by CRISPR-Cas9 gene editing using ribonucleoprotein complex (RNP). NK cells are then further expanded in vitro and on day 14 functional studies, mass cytometry and RNA sequencing are performed as needed.

FIG. 2. Schematic diagram depicting an example of a protocol for CAR transduction and CRISPR Cas9 gene editing of primary NK cells, such as in the GMP.

FIGS. 3A-3C. Comparison of small- and large-scale efficient CRISPR RNP mediated KO of CD47 in primary CAR-NK cells. FIG. 3A. Electroporation efficiency demonstrated by tracrRNA fluorescent label at 5 million and 30 million scale levels. FIG. 3B. Knockout efficiency shown by PCR gel electrophoresis at 5 million and 30 million electroporated cell dose levels. FIG. 3C. Knockout efficiency shown by flow cytometry again at the small- and large-scale levels.

FIGS. 4A-4F. Comparison of small and large-scale CRISPR RNP mediated KO of TGFBR2 in primary CAR-NK cells. FIG. 4A. Electroporation efficiency demonstrated by tracrRNA fluorescent label at 5 million and 30 million scale levels. FIG. 4B. Knockout efficiency shown by PCR gel electrophoresis in 5 million vs. 30 million cells. FIG. 4C. Total nucleated live cell counts showing live cell numbers compared to Cas9 control following RNP mediated KO at 5 million vs. 30 million cells. FIG. 4D. Phosphoflow assay to assess pSMAD2/3 signaling with or without treatment with TGF-beta at both the 5 million and 30 million dose levels. FIG. 4E. Apoptosis Annexin V assay gated on THP-1 tumor targets, at both the 5 million and 30 million cell KO scales. Lonza 4-D; 100 ul X kit L-V4XP-3024; 1 ml LV Unit-V4LP-3002; Program EO-115; Molar Ratio of Cas9/gRNA-4.6uM gRNA,4uMCas9. FIG. 4F. Heatmap summarizing phenotyping using mass cytometry for cas9 control or TGFBR2 KO CAR NK cells at the 5 million and 30 million cell KO scale levels.

FIGS. 5A-5L. Examples of sequences of the crRNAs used for each targeted gene with a representative example of KO efficiency depicted by either flow cytometry or PCR. FIG. 5A. NKG2A (Exon 4), SEQ ID NO:1; FIG. 5B. TIM3 (Exon 2), SEQ ID NO:2; FIG. 5C. SHP1 (Exon 4; SEQ ID NO:3; FIG. 5D. TIGIT (Exon 3), SEQ ID NO:4; FIG. 5E. CD96 (Exon 2), SEQ ID NO:5; FIG. 5F. TGFBR2 (Exon 5), SEQ ID NO: 6 for gRNA1 and SEQ ID NO:7 for gRNA2; FIG. 5G. CISH (Exon 4), SEQ ID NO:8 for crRNA1 and SEQ ID NO:9 for crRNA2; FIG. 5H. PD1 (Exon 2), SEQ ID NO:10; FIG. 5I. A2AR (Exon 3), SEQ ID NO:11 for crRNA1 and SEQ ID NO:12 for crRNA2; FIG. 5J. LAG3 (Exon 3), SEQ ID NO:13; FIG. 5K. Siglec 7 (Exon 1), SEQ ID NO:14; FIG. 5L. CD38 (Exon 2), SEQ ID NO:15.

FIG. 6. Examples of sequences of the crRNAs used for each targeted gene.

FIGS. 7A-7C. CRISPR/Cas9 mediates efficient multiple genes disruption in NK cells. FIGS. 7A-7B. PCR gel electrophoresis showing the KO efficiency of the different genes (in 2 different sets, set 1 in FIG. 7A panel and set 2 in FIG. 7B panel) in 2 NK cell donors compared to wild type (WT) on the left panels. FIG. 7C. Histograms showing the KO efficiency of the various genes by flow cytometry compared to Cas9 control.

FIGS. 8A-8G. Multiplex gene editing of inhibitory molecules in NK cells improves their in vitro functionality. FIG. 8A. Representative FACS plots showing increased cytokine production (IFNg, TNFa) and degranulation (CD107a) by CAR19-NK cells following triple KO of NKG2A, TGFBR2 and CISH compared to Cas9 control when tested against Raji lymphoma. FIG. 8B. Bar graph summarizing the cytokine production and degranulation data from functional assays of 3 different NK cell donors. FIG. 8C. Chromium release assay showing increased specific lysis of Raji tumor cells by triple KO CAR19-NK cells compared to Cas9 control. FIG. 8D. Incucyte killing assay showing enhanced killing of Raji tumor cells by triple KO CAR19-NK cells compared to Cas9 control. FIG. 8E. Representative FACS plot of Annexin V apoptosis assay showing increased apotosis of Raji tumor after co-culture with triple KO CAR19-NK cells compared to Cas9 control. FIG. 8F. Bar graph summarizing the data of annexin V apoptosis assay from 3 NK cell donors. FIG. 8G. Bar graph showing increased Perforin and Granzyme B production by CAR19-NK cells following triple KO of NKG2A, TGFBR2 and CISH compared to Cas9 control when tested against Raji lymphoma

FIGS. 9A-9B. Multiplex gene editing of inhibitory molecules in NK cells improves their in vivo anti-tumor activity in a Raji lymphoma mouse model. FIG. 9A. BLI imaging showing improved tumor control in the group of mice that received Raji+ triple KO CAR19-NK cells compared to the groups that received Raji+Cas9 control CAR19-NK cells or Raji alone. FIG. 9B. Kaplan Meir curves showing enhances survival of mice that received Raji+ triple KO CAR19-NK cells compared to the groups that received Raji+Cas9 control CAR19-NK cells or Raji alone.

FIGS. 10A-10C. ADAM17 KO in CAR-NK cells prevents shedding of CD16 and CD62L. FIG. 10A. PCR DNA gel electrophoresis showing efficient KO of ADAM17 in CAR-NK cells. FIG. 10B. Representative FACS plots showing increased expression of CD16 on CAR-NK cells following ADAM17 KO with or without co-culture with tumor and with or without treatment with Rituximab (a monoclonal antibody for targeting CD20-expressing cancers). FIG. 10C. Representative FACS plots showing increased CD62L expression on CAR-NK cell following ADAM17 KO as compared with WT NK cells with or without co-culture with tumor.

FIGS. 11A-11B. demonstrate NR3C1 knockout efficiency being tested using PCR for various programs (CM137, DN100, CA137, DS137 and EH100) for electroporation in Lonza 4D-Nucleofactor™. This was performed in small scale, with 5E6 T cells and Plasmalyte (supplemented with HEPES and mannitol, PL); FIG. 11A shows knockout of the NR3C1 gene in the T cells using the various manufacturer programs. FIG. 11B shows fold expansion of NR3C1 knockout cells tested with various electroporation programs.

FIGS. 12A-12B. concern NR3C1 knockout efficiency using PCR tested for the CM137 program used for electroporation in Lonza 4D-Nucleofactor™. This was done in large scale, with 50E6, using either Lonza buffer (P3) or PL (supplemented with HEPES and mannitol) and with 100E6 using PL (supplemented with HEPES and mannitol) (FIG. 12A). FIG. 12B shows NR3C1 knockout efficiency using CA137 program for electroporation in Lonza 4D-Nucleofactor™. This was done in large scale, with either 50E6 or 100E6 using PL (supplemented with HEPES and mannitol).

### DESCRIPTION OF ILLUSTRATIVE EMBODIMENTS

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they have reduced expression of one or more endogenous genes and/or because they express one or more heterologous genes (such as synthetic antigen receptors and/or cytokines), in which case(s) the engineering is all performed by the hand of man. With respect to an antigen receptor, the antigen receptor may be considered engineered because it comprises multiple components that are genetically recombined to be configured in a manner that is not found in nature, such as in the form of a fusion protein of components not found in nature so configured.

The term “large-scale” as used herein refers to on the order of up to 10⁹ or more, including 10¹⁰, 10¹¹, and so forth number of NK cells.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” or “individual” refer to either a human or non-human, such as primates, mammals, and vertebrates. In particular embodiments, the subject is a human.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, horses, goats, sheep, and chimpanzees. Mammals may be referred to as “patients” or “subjects” or “individuals”.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “heterologous” as used herein refers to being derived from a different cell type or a different species than the recipient. In specific cases, it refers to a gene or protein that is synthetic and/or not from an NK cell. The term also refers to synthetically derived genes or gene constructs. The term also refers to synthetically derived genes or gene constructs. For example, a cytokine may be considered heterologous with respect to a NK cell even if the cytokine is naturally produced by the NK cell because it was synthetically derived, such as by genetic recombination, including provided to the NK cell in a vector that harbors nucleic acid sequence that encodes the cytokine.

The present disclosure concerns a novel approach for the large-scale expansion, CAR transduction, cytokine expression and gene editing of NK cells. This approach allows expansion of NK cells (for example, peripheral blood-derived, cord blood-derived, stem cell-derived (e.g., hematopoietic or induced pluripotent stem cells), or cell line-derived NK cells) to be expanded to large numbers and transduced to redirect their specificity against one or more tumor antigens, in addition to optionally expressing one or more heterologous antigen receptors and/or one or more heterologous cytokine genes. The function of NK cells is further improved by deleting gene(s) that increase activity of the NK cells, such as one or more genes involved in cell exhaustion and tumor-induced dysfunction, as some examples.

In particular embodiments, the disclosure is the first to report that combined CAR transduction and deletion of single or multiple genes in human NK cells contributes to the cell's improved function and resistance to the tumor microenvironment. In particular, a large-scale and GMP grade protocol is used for this approach for translation to a clinic. The disclosure has direct implications on patient care using a novel immunotherapeutic approach that enhances the function of a patient's own NK cells or adoptively transferred NK cells, or a mixture thereof.

### II. Process

Embodiments of the disclosure concern processes of generating engineered NK cells, particularly on a large scale, and compositions comprising, consisting of, or consisting essentially of products produced therefrom. In specific embodiments, the process utilizes one or a combination of particular parameters to produce certain types of engineered NK cells where the parameters may include certain concentrations of reagents, certain types of NK cells, certain durations of time for one or more steps, certain types of modifications, certain types of cell modification mechanisms, or a combination thereof. The skilled artisan will recognize that although optimum variables are described herein, there are also variations of the process that will still produce a suitable amount of effective engineered NK cells, and these are also encompassed herein.

In particular embodiments, the process concerns multiple steps where cells are generated by modification for them to express one or more heterologous proteins but also by modification to their genome by gene editing to have reduced or eliminated expression of one or more endogenous genes for the cells. In some cases, the cells may be subject to selection against one or more markers or selection for one or more markers. The steps may or may not have a particular order. In particular embodiments, one or more steps occur concurrently, and/or one or more steps occur successively. In specific cases, the process generally concerns a succession of steps, and in specific embodiments the succession comprises expansion of NK cells, engineering of the NK cells in two or more aspects, and expansion of the produced engineered cells, followed by an optional analysis step and/or an optional administration step to an individual. In specific embodiments, the engineering includes both of (1) modifying the cells to express a heterologous protein, such as an antigen receptor, and (2) modifying the cells to have reduced expression (knockdown) or elimination of expression (knockout) of one or more endogenous genes in the NK cells. The genes may be specifically selected for knockdown or knockout because in doing so, the resultant modified cells have one or more enhanced activities compared to NK cells that are not so modified. Examples of such activities include anti-tumor activity, potency, proliferation, trafficking, antibody-dependent cellular cytotoxicity, resistance to corticosteroids, and/or persistence, for example. In some cases (1) occurs before (2), whereas in other cases (2) occurs before (1). Although the modifying of the cells to have reduced expression or eliminated expression may occur by any means, in particular embodiments the modifying is by CRISPR.

An initial step for the process may include the expansion of NK cells that allows an increase in number of NK cells for eventual modification. The NK cells may be obtained from a fresh source or from storage or commercially, for example. The NK cells can come from a donor or an individual in need of the ultimate modified NK cells. Although the NK cells may be of any kind, in specific embodiments the NK cells are derived from cord blood, peripheral blood, bone marrow, hematopoietic or induced pluripotent stem cells, NK cell lines, or a mixture thereof. In particular cases the NK cells are from cord blood. In particular embodiments, the starting culture of NK cells for the expansion step includes at least 5-100 million cells, and in some cases 1×10⁹ cells are used. Thus, in specific cases the number of NK cells for initiating the procotol may be 5, 10, 15, 20, 25, 30, 35, 40, 45, 50, 55, 60, 65, 70, 75, 80, 85, 90, 95, 100 million, or more NK cells. A range of cells may be utilized at any step of the process, including 5-100, 5-90, 5-75, 5-50, 5-25, 5-10, 10-100, 10-90, 10-75, 10-60, 10-50, 10-25, 25-100, 25-90, 25-75, 25-50, 50-100, 50-90, 50-75, 75-100, 75-90, or 90-100 million cells. In some embodiments, a range of cells includes up to 1×10⁸, 1×10⁹, 1×10¹⁰, and so forth.

In particular embodiments, prior to initiation of the expansion step in culture, the NK cells are subject to depletion of particular undesirable cells that may be present with a population of NK cells, such as T cells, B cells, and/or monocytes. The depletion step may exploit the presence of certain markers on the undesirable cells as a means to cull them from the population of NK cells. For example, in specific cases the starting source of NK cells is subject to depletion of cells positive for CD3, CD14, and/or CD19. As one example of a means to do this, the cells may be subject to depletion using immunomagnetic beads (having antibodies to CD3, CD14, and/or CD19, respectively, attached thereto), thereby producing an enriched population of NK cells. In other cases, the desired NK cells from a mixed population can be selected for by the presence of one or more markers on the NK cells, such as CD56, CD16, NKp46, and/or NKG2D.

The expansion step may or may not begin with an enriched population of NK cells being counted to provide an accurate assessment of quantity for culturing of the NK cells with feeder cells, such as antigen presenting cells that may or may not be irradiated. The ratio of NK cells to APCs in the expansion step may be of a certain number, such as 1:1, 1:1.5, 1:2, or 1:3, for example. In certain aspects, the APCs are engineered to express membrane-bound IL-21 (mbIL-21). In particular aspects, the APCs are alternatively or additionally engineered to express IL-21, IL-15, and/or IL-2. In some aspects, the APCs are universal antigen presenting cells (uAPCs). In certain aspects, the uAPCs are engineered to express (1) CD48 and/or CS1 (CD319), (2) membrane-bound interleukin-21 (mblL-21), and/or (3) 41BB ligand (41BBL). In some aspects, the uAPCs express CD48. In certain aspects, the uAPCs express CS1. In particular aspects, the uAPCs express CD48 and/or CS1. In some aspects, the uAPCs have essentially no expression of endogenous HLA class I, II, and/or CD1d molecules. In certain aspects, the uAPCs express ICAM-1 (CD54) and/or LFA-3 (CD58). In particular aspects, the uAPCs are further defined as leukemia cell-derived aAPCs, such as K562 cells.

In particular embodiments, the media in which the expansion step(s) occurs comprises one or more agents to facilitate expansion, such as one or more cytokines. In specific embodiments, the media comprises IL-2, IL-15, IL-18, and/or IL-21 and may not comprise other exogenously added cytokines. In some embodiments, the concentration of IL-2 in the media is 10-1000 IU, including 10-1000, 10-750, 10-500, 10-250, 10-200, 10-100, 50-1000, 50-750, 50-500, 50-250, 50-200, 50-100, 100-1000, 100-750, 100-500, 100-250, 100-200, 250-1000, 250-750, 250-500, 500-1000, 500-750, or 750-1000 IU. In specific embodiments, the concentration of IL-2 in the media is in the range of 100-300 units/mL, including 100, 125, 150, 175, 200, 225, 250, 275, or 300 units/mL. The expansion step may occur at a certain temperature, such as about 35° C.-38° C., including at 35° C., 36° C., 37° C., or 38° C. Different expansion steps may or may not occur at different temperatures. The expansion step may or may not occur at a certain level of oxygen, such as from 3%-7% CO₂; in specific embodiments the expansion step occurs at 3%, 4%, 5%, 6%, or 7% CO₂. Different expansion steps may or may not occur at different oxygen levels. The expansion step may last for a particular duration of time, such as a certain number of hours or days. In specific embodiments, an expansion step lasts for 4, 5, 6, 7, or more days, but in specific cases the expansion step lasts from 4-7, 4-6, 4-5, 5-6, 5-7, or 6-7 days. Different expansion steps may or may not occur at different durations of time. In particular embodiments, the media in the expansion step may or may not be changed during the expansion, but in specific embodiments on day 3 after initiation of the expansion step the media is changed, such as replaced with generally the same content as the original media. In specific cases the cells are centrifuged and resuspended in the same or a similar media than before, including with a media comprising 100, 125, 150, 175, 200, 225, 250, 275, or 300 units/mL of IL-2. In specific aspects, the expansion step occurs in a bioreactor, such as a gas permeable bioreactor, for example G-Rex®100M or G-Rex100®. In certain aspects, the bioreactor is a gas permeable bioreactor. In particular aspects, the gas permeable bioreactor is G-Rex®100M or G-Rex100®. In some aspects, the stimulating of step (b) is performed in a certain volume of media, such as 3-5 L of media, such as 3, 3.5, 4, 4.5, or 5 L.

Following a certain number of days after initiation of the expansion step, the cells may be further subjected to a depletion step, although in alternative embodiments they are not subjected to a depletion step. In specific cases, on days 5 or 6 after initiation of the expansion step, the cells are subjected to a depletion step for depletion of cells positive for CD3, CD14, and/or CD19.

On about day 3, 4, 5, 6, 7 or 8 following initiation of expansion of the NK cells, the expanded NK cells may be subjected to one or more modifications. The first modification may be transduction or transfection of the NK cells to express a heterologous antigen receptor, although in some cases the first modification is disrupting expression of one or more endogenous genes of the NK cells. When the first modification is transduction or transfection of the NK cells to express a heterologous antigen receptor, a subsequent modification is disrupting expression of one or more endogenous genes of the NK cells. When the first modification is disrupting expression of one or more endogenous genes of the NK cells, a subsequent modification is transduction or transfection of the NK cells to express a heterologous antigen receptor.

In specific embodiments, the expanded NK cells are transduced or transfected to harbor a heterologous antigen receptor gene prior to the cells being gene edited for disruption of expression of one or more endogenous genes. In particular embodiments, the cells are transduced or transfected with a particular vector that comprises an expression construct that encodes one or more chimeric antigen receptors, one or more T cell receptors, or a combination thereof. The vector may be of any kind including at least nanoparticles, plasmid, lentiviral vector, retroviral vector, adenoviral vector, adenoviral-associated vector, and so forth. The NK cells may be transfected or transduced with a vector that allows for the NK cells to express multiple heterologous proteins, such as a heterologous antigen receptor(s), a suicide gene, and one or more cytokines, such as one or more heterologous cytokines selected from the group consisting of IL-7, IL-2, IL-15, IL-12, IL-18, IL-21, and a combination thereof. Although genes for the multiple heterologous proteins may be present on the same vector, in some cases they are present on multiple vectors. Once the cells have been transduced or transfected, the NK cells are modified and may or may not be tested for expression of the heterologous protein(s), and this may occur 1, 2, 3, or more days following transfection/transduction. When testing an aliquot from the population of modified NK cells, the testing may or may not occur prior to further modification, such as prior to gene editing of the NK cells.

In some embodiments, within about day 6, 7, 8, 9, 10, 11, or 12 following initiation of expansion of the NK cells, the modified NK cells may be subjected to methods of gene editing. The gene editing step may or may not occur within 1, 2, 3, or more days following the transfection/transduction step. The gene editing of the modified NK cells may cocur by any suitable method, but in particular embodiments, the gene editing of the modified NK cells occurs by CRISPR methods. Thus, in particular embodiments the modified cells are exposed to suitable amounts of Cas9 and guide RNA. In cases wherein more than one gene of the NK cells is to be disrupted for expression, there may be a group of guide RNAs that includes one or more sequence-specific guide RNAs for each of the desired genes to be edited. In particular embodiments, and as an example wherein two endogenous genes in the NK cells are to be edited, the modified NK cells are subjected to two different electroporation steps separated in time by 1, 2, 3, or more days. In some cases, a first electroporation step comprises targeting one, two, or more genes and a second electroporation step comprises targeting one, two, or more genes that are different genes than in the first electroporation step. In some cases, there are successive electroporation steps beyond two electroporation steps, including 3, 4, 5, or more additional electroporation steps. In any case wherein multiple electroporation steps are utilized, the next electroporation may occur only after a duration of time, such as only after 1, 2, 3, 4, or more days since the prior electroporation step.

In certain embodiments, the NK cells undergo the gene editing step first, followed by the transduction or transfection step to harbor a heterologous antigen receptor gene.

Following gene editing and transformation/transduction of the NK cells, the NK cells may or may not be subjected to a second expansion step to increase the number of modified gene edited NK cells. In specific embodiments, the second expansion step in the process may be substantially identical to the first expansion step in the process, although in alternative embodiments the second expansion step is different than the first expansion step, such as having different media, different exogenously added compounds, different duration of time for culture/expansion, different expansion flasks such as GREX or WAVE to name a few, or a combination thereof, and so forth. In specific cases, the second expansion step comprises culturing of the modified gene edited NK cells with feeder cells, including antigen presenting cells that may or may not be irradiated. In this second expansion step, the ratio of NK cells to APCs may be of a certain number, such as 1:1, 1:1.5, 1:2, or 1:3, for example. In particular embodiments, the media in which the expansion step occurs comprises one or more agents to facilitate expansion, such as one or more cytokines. In specific embodiments, the media comprises IL-2 and may not comprise other exogenously added cytokines. In specific embodiments, the concentration of IL-2 in the media is in the range of 100-300 units/mL, including 100, 125, 150, 175, 200, 225, 250, 275, or 300 units/mL. The expansion step may occur at a certain temperature, such as about 35° C-38° C., including 35° C., 36° C., 37° C., or 38° C. The expansion step may occur at a certain level of oxygen, such as from 3%-7% CO₂; in specific embodiments the expansion step occurs at 3%, 4%, 5%, 6%, or 7% CO₂. The expansion step may last for a particular duration of time, such as a certain number of days. In specific embodiments, the expansion step lasts for 4, 5, 6, 7, or more days, but in specific cases the expansion step lasts from 4-7, 4-6, 4-5, 5-6, 5-7, or 6-7 days. In particular embodiments, the media in the expansion step may or may not be changed during the expansion, but in specific embodiments on day 3 after initiation of the expansion step the media is changed. In specific cases the cells are centrifuged and resuspended in the same or a similar media than before, including with a media comprising 100, 125, 150, 175, 200, 225, 250, 275, or 300 units/mL of IL-2. In at least some cases, the method is performed in a bioreactor, including a gas permeable bioreactor. In particular aspects, the gas permeable bioreactor is G-Rex®100M or G-Rex100®.

Following a specific duration of time for the second expansion step, the cells may be utilized, analyzed, stored, and so forth. For example, the cells may be analyzed for (1) the ability of the heterologous antigen receptor to bind its target antigen; (2) expression, or lack thereof, of the edited gene(s) to confirm knockdown or knockout of expression; or (3) both. In specific embodiments, the cells are analyzed for functionality, cytotoxicity, in vivo activity, expression status, cytokine production, degranulation, and so forth. Analysis methods may include PCR, flow cytometry, mass cytometry, RNA sequencing, Incucyte killing, BLI imaging, chromium release assay, Annexin V apoptosis assay, or a combination thereof.

In particular embodiments, the produced NK cells are stored, including cryopreserved, for example. For example, the NK cells may be cryopreserved for use by the individual from which the starting NK cells were obtained, or the NK cells may be cryopreserved for use by an individual that is different than the individual from which the starting NK cells were obtained.

### III. NK Cells

The present disclosure concerns processes of modifying NK cells, as opposed to other types of immune cells. NK cells are a subpopulation of lymphocytes that have spontaneous cytotoxicity against a variety of tumor cells, virus-infected cells, and some normal cells in the bone marrow and thymus. NK cells differentiate and mature in the bone marrow, lymph nodes, spleen, tonsils, and thymus. NK cells can be detected by specific surface markers, such as CD16 and/or, CD56 in humans. NK cells do not express T cell antigen receptors, the pan T marker CD3, or surface immunoglobulin B cell receptors.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood or NK cell lines by methods well known in the art. Particularly, umbilical CB may be used to derive NK cells. In certain aspects, the NK cells are isolated and expanded by the previously described method of ex vivo expansion of NK cells (Spanholtz et al., 2011; Shah et al., 2013). In this method, CB mononuclear cells are isolated by ficoll density gradient centrifugation and cultured in a bioreactor with IL-2 and artificial antigen presenting cells (aAPCs). After 7 days, the cell culture may be depleted of any cells expressing CD3 and re-cultured for an additional 7 days. The cells may again be CD3-depleted and characterized to determine the percentage of CD56⁺/CD3⁻ cells or NK cells. In other methods, umbilical CB is used to derive NK cells by the isolation of CD34⁺ cells and differentiation into CD56⁺/CD3⁻ cells by culturing in medium contain SCF, IL-7, IL-15, and/or IL-2.

As described elsewhere herein, the NK cells are expanded once or twice during their preparation. As noted above, in specific cases expansion of the NK cells comprises: stimulating mononuclear cells (MNCs) from cord blood in the presence of antigen presenting cells (APCs) and IL-2; and at some point in the process re-stimulating the cells with APCs to produce expanded NK cells. In at least some cases, the method is performed in a bioreactor. Multiple steps of the process may occur in the same vessel, including the same bioreactor. The stimulating step can direct the MNCs towards NK cells. The re-stimulating step may or may not comprise the presence of IL-2. In particular aspects, the method does not comprise removal or addition of any media components during a stimulating step. In particular aspects, the method is performed within a certain time frame, such as in less than 15 days, for example in 14 days.

As detailed elsewhere herein, in a certain embodiment, the NK cells are expanded by an ex vivo method for the expansion comprising: (a) obtaining a starting population of mononuclear cells (MNCs) from cord blood; (b) stimulating the MNCs in the presence of antigen presenting cells (APCs) and IL-2; and (c) re-stimulating the cells with APCs to produce expanded NK cells, wherein the method is performed in a bioreactor and is good manufacturing practice (GMP) compliant. The stimulating of step (b) can direct the MNCs towards NK cells. Step (c) may or may not comprise the presence of IL-2, in some cases. In particular aspects, the method does not comprise removal or addition of any media components during step (b). In particular aspects, the method is performed in less than 15 days, such as in 14 days.

As described, the method further comprises depleting cells positive for one or more particular markers, such as CD3, CD14, and/or CD19, for example. In certain aspects, the depleting step is performed prior to transduction or transfection and/or after gene editing. In some aspects, the cells are removed from the bioreactor for CD3, CD14, and/or CD19 depletion and placed in the bioreactor for subsequent steps.

In certain aspects, obtaining the starting population of MNCs from cord blood comprises thawing cord blood in the presence of dextran, human serum albumin (HSA), DNAse, and/or magnesium chloride. In particular aspects, obtaining the starting population of MNCs from cord blood comprises thawing cord blood in the presence of dextran and/or DNase. In specific aspects, the cord blood is washed in the presence of 5-20%, such as 10%, dextran. In certain aspects, the cord blood is suspended in the presence of magnesium chloride, such as at a concentration of 100-300 mM, particularly 200 mM. In some aspects, obtaining comprises performing ficoll density gradient centrifugation to obtain mononuclear cells (MNCs). In certain aspects, the cord blood from which the NK cells are derived is frozen cord blood. In particular aspects, the frozen cord blood has been tested for one or more infectious diseases, such as hepatitis A, hepatitis B, hepatitis C, Trypanosoma cruzi, HIV, Human T-Lymphotropic virus, syphyllis, Zika virus, and so forth. In some aspects, the cord blood is pooled cord blood, such as from 3, 4, 5, 6, 7, or 8 individual cord blood units.

In certain aspects, the method does not comprise human leukocyte antigen (HLA) matching. In some aspects, the starting population of NK cells are not obtained from a haploidentical donor.

In some aspects, the expanded NK cells produced from the process comprise a clinically relevant dose. In some aspects, the NK cells are autologous with respect to a recipient individual. In certain aspects, the NK cells are allogeneic with respect to a recipient individual.

### IV. Heterologous Antigen Receptors

The NK cells of the present disclosure can be genetically engineered to express one or more heterologous antigen receptors, such as one or more engineered TCRs, one or more CARs, one or more chimeric cytokine receptors, one or more chemokine receptors, a combination thereof, and so on. The heterologous antigen receptors are synthetically generated by the hand of man. In particular embodiments, the NK cells are modified to express one or more CAR and/or one or more TCR, each having antigenic specificity for a different cancer antigen. Multiple CARs and/or TCRs, such as directed toward different antigens, may be added to the NK cells. In some aspects, the immune cells are engineered to express the CAR(s) and/or TCR(s) by knock-in of the CAR or TCR at a particular gene locus, such as by using CRISPR.

Although in particular cases the NK cells are particularly edited using CRISPR, alternative suitable methods of modification are known in the art. See, for instance, Sambrook and Ausubel, supra. For example, the cells may be transduced to express a CAR or TCR having antigenic specificity for a cancer antigen using transduction techniques described in Heemskerk et al., 2008 and Johnson et al., 2009. In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Patent No.: 7,446,190, and those described in International Patent Application Publication No.: WO/2014055668 A1.

A. Chimeric Antigen Receptors

In some embodiments, the CAR comprises: a) one or more intracellular signaling domains, b) a transmembrane domain, and c) an extracellular domain comprising one or more antigen binding regions.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, 4-1BB (CD137), or a combination thereof. In addition to a primary signal initiated by CD3ζ, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of antigens include CD19, CD70, HLA-G, CD38, CD123, CLL1, EBNA, CD123, HER2, CA-125, TRAIL/DR4, CD20, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B 1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1, or a combination thereof.

In certain embodiments, the CAR may be co-expressed with one or more cytokines to improve persistence when there is a low amount of tumor-associated antigen. For example, CAR may be co-expressed with one or more cytokines, such as IL-7, IL-2, IL-15, IL-12, IL-18, IL-21, or a combination thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

B. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine -based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

C. Antigen-Presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

D. Antigens

Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, EBNA, CD123, HER2, CA-125, TRAIL/DR4, CD20, CD70, HLA-G, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STATS, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B 1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1. Examples of sequences for antigens are known in the art, for example, in the GenBank® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, instead of a cancer antigen (tumor antigen), an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

E. Suicide Genes

In some cases, any cells of the disclosure are modified to produce one or more agents other than heterologous cytokines, engineered receptors, and so forth. In specific embodiments, the cells, such as NK cells, are engineered to harbor one or more suicide genes, and the term “suicide gene” as used herein is defined as a gene which, upon administration of a prodrug, effects transition of a gene product to a compound which kills its host cell. In some cases, the NK cell therapy may be subject to utilization of one or more suicide genes of any kind when an individual receiving the NK cell therapy and/or having received the NK cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Examples of suicide genes include engineered nonsecretable (including membrane bound) tumor necrosis factor (TNF)-alpha mutant polypeptides (see PCT/US19/62009, which is incorporated by reference herein in its entirety), and they may be targeted by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP), as examples.

F. Methods of Delivery

In specific embodiments, any composition may be delivered to the recipient NK cells by any suitable methods. The compositions may be delivered to the NK cells by electroporation or by a vector, for example. In specific embodiments, for example, one or more compositions for introduction of at least one or more heterologous antigen receptors are delivered to the NK cells in a vector. In some embodiments, one or more compositions for gene editing are delivered to the NK cells in a vector. One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

In specific embodiments, the vector is a multicistronic vector, such as is described in PCT/US19/62014, which is incorporated by reference herein in its entirety. In such cases, a single vector may encode the CAR or TCR (and the expression construct may be configured in a modular format to allow for interchanging parts of the CAR or TCR), a suicide gene, and one or more cytokines.

1. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

a. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

b. Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCRTM, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD1 lc, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

c. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth diease virus 2A) or a “2A-like” sequence (e.g., Thosea asigna virus 2A; T2A).

d. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

e. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

2. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### V. Gene Editing and CRISPR

The NK cell production process of the disclosure may include gene editing of the NK cells to remove 1, 2, 3, 4, 5, 6, 7, 8, 9, 10, or more endogenous genes in the NK cells. In some cases the gene editing occurs in NK cells expressing one or more heterologous antigen receptors, whereas in other cases the gene editing occurs in NK cells that do not express a heterologous antigen receptor but that ultimately will express one or more heterologous antigen receptors, in at least some cases. In particular embodiments, the NK cells that are gene edited are expanded NK cells.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. Iin specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, and CD39. In specific embodiments, the TGFBR2 gene is knocked out or knocked down in the NK cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### VI. Methods of Treatment

In some embodiments, the NK cells produced by the mehods of the disclosure are utilized for methods of treatment for an individual in need thereof. Embodiments of the disclosure include methods of treating an individual for cancer, infections of any kind, and/or any immune disorder, as examples. The individual may utilize the treatment method of the disclosure as an initial treatment or after (and/or with) another treatment. In cancer embodiments, the immunotherapy methods may be tailored to the need of an individual with cancer based on the type and/or stage of cancer, and in at least some cases the immunotherapy may be modified during the course of treatment for the individual.

In specific cases, examples of treatment methods are as follows: 1) Adoptive cellular therapy with the produced NK cells (ex vivo expanded or expressing CARs or TCRs) to treat cancer patients with any type of hematologic malignancy, (2) Adoptive cellular therapy with the produced NK cells (ex vivo expanded or expressing CARs or TCRs) to treat cancer patients with any type of solid cancers, (3) Adoptive cellular therapy with the produced NK cells (ex vivo expanded or expressing CARs or TCRs) to treat patients with infectious diseases and/or immune disorders.

In some embodiments, the present disclosure provides methods for immunotherapy comprising administering an effective amount of the NK cells produced by methods of the present disclosure. In one embodiment, a medical disease or disorder is treated by one or more transfers of NK cell populations produced by methods herein and that elicit an immune response, in at least particular cases. In certain embodiments of the present disclosure, cancer or infection is treated by delivery of one or more NK cell populations produced by methods of the disclosure and that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and/or viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of hematological malignancies, such as lymphoma or leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days, or 1, 2, 3, or 4 or more weeks, or 1, 2, 3, 4, 5, 6, 7, 8, 9, 10, 11, 12, or more months.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, one or more growth factors that promotes the growth and activation of the NK cells is administered to the subject either concomitantly with the NK cells or subsequently to the NK cells. The growth factor can be any suitable growth factor that promotes the growth and activation of the NK cells. Examples of suitable immune cell growth factors include interleukin (IL)-2, IL-7, IL-12, IL-15, IL-18, and IL-21, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of the produced NK cells can be administered by a number of routes, including parenteral administration, for example, intravenous, intraperitoneal, intramuscular, intrasternal, intratumoral, intrathecal, intraventricular, through a reservoir, intraarticular injection, or infusion.

The therapeutically effective amount of the produced NK cells for use in adoptive cell therapy is that amount that achieves a desired effect in a subject being treated. For instance, this can be the amount of NK cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The produced NK cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of NK cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ NK cells/m². In a certain embodiment, the dose used in the treatment of human subjects ranges from about 3.8×10⁹ to about 3.8×10¹⁰ NK cells/m². In additional embodiments, a therapeutically effective amount of NK cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of NK cells is readily determined by one of skill in the art based on the age, weight, sex, and physiological condition of the subject. Effective doses can be extrapolated from dose-response curves derived from in vitro or animal model test systems.

The NK cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

A. Pharmaceutical Compositions

Also provided herein are pharmaceutical compositions and formulations comprising NK cells produced by the processes encompassed herein and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve a NK cell population in combination with at least one additional therapy. For cancer embodiments, the additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. For pathogenic conditions, the additional therapy may comprise one or more antibiotics, antivirals, and so forth.

In some cancer embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

A NK cell therapy of the disclosuremay be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs inlcude ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. Articles of Manufacture or Kits

An article of manufacture or a kit is provided comprising NK cells, and/or one or more reagents for generating them. The NK cells may be from any source, and in specific embodiments the NK cells have been produced by methods encompassed herein. In specific embodiments, the NK cells have been gene edited and may be provided in the kit so that they may be further modified to express one or more heterologous antigen receptors. In specific embodiments, the NK cells have been modified to express one or more heterologous antigen receptors and may be provided in the kit so that they may be further modified to be gene edited. In specific embodiments, one or more reagents for generating the NK cells are provided in the kit, such as reagents that target a specific NK cell gene, reagents that comprise a heterologous antigen receptor (or one or more reagents to produce the heterologous antigen receptor), or a combination thereof. In general embodiments, the reagents may comprise nucleic acid including DNA or RNA, protein, media, buffers, salts, co-factors, and so forth. In specific cases, the kit comprises one or more CRISPR-associated reagents, including for targeting a specific desired NK cell gene.

The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Large-Scale Combined CAR Transduction and CRISPR Gene Editing of NK Cells

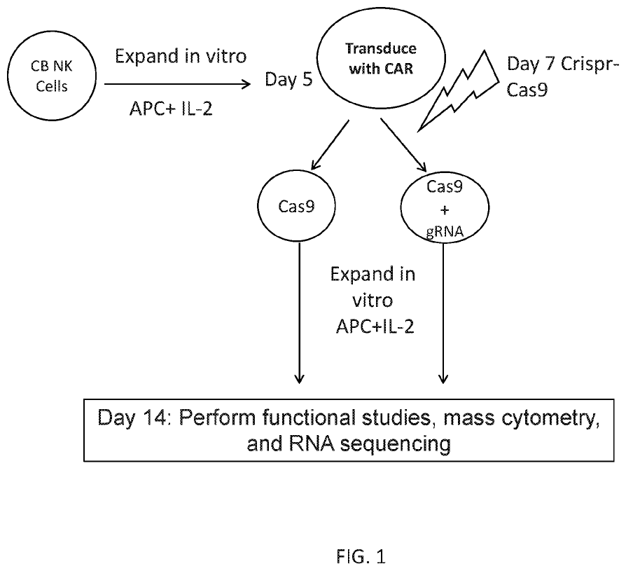

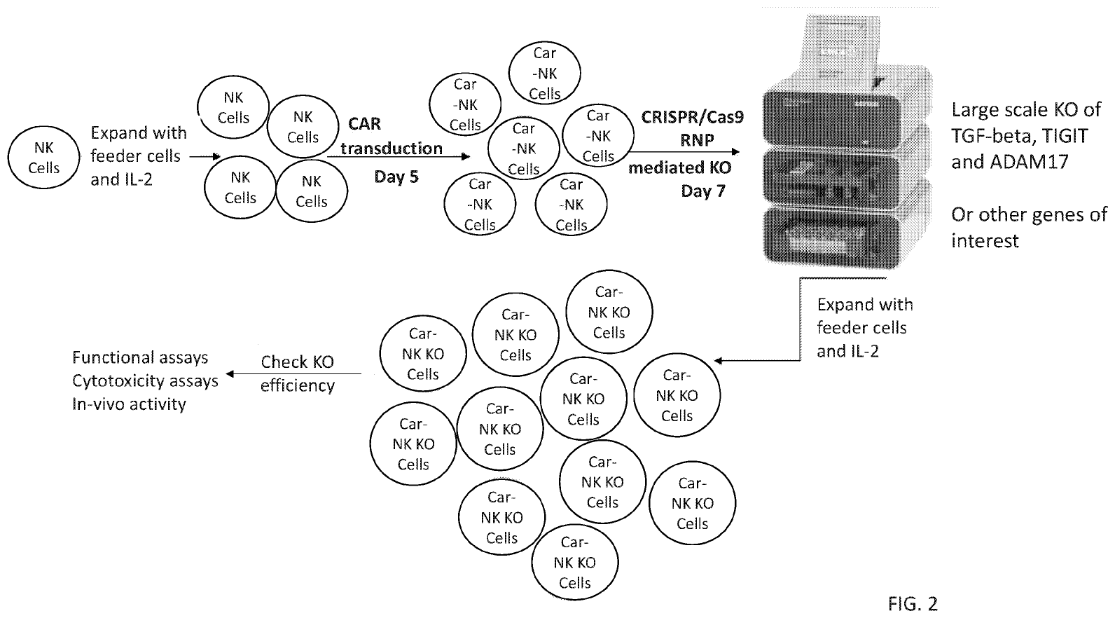

The present example concerns production of NK cells for immunotherapy using a large-scale process that also combines transduction of the NK cells with at least one CAR molecule and CRISPR gene editing of at least one endogenous gene in the NK cells. As one example, illustrated FIG. 1, there is a protocol combining CAR transduction and CRISPR Cas9 gene editing of primary cord blood-derived NK cells. In this example, CB NK cells are expanded with feeder cells (APCs) and IL-2. On day 5 of culture, the NK cells are transduced with a CAR construct via retroviral vector. On day 7, this is followed by CRISPR-Cas9 gene editing using ribonucleoprotein complex (RNP) (Cas9 alone as control in the circle on the left and those electroporated with both gRNA and Cas9 (RNP complex) in the circle on the right). NK cells are then further expanded in vitro with APCs and IL-2 and on day 14 the cells may be analyzed, such as with one or more of functional studies, mass cytometry and RNA sequencing. FIG. 2 provides a schematic diagram illustrating one example of a protocol for CAR transduction and CRISPR Cas9 gene editing of primary NK cells in the GMP. NK cells are expanded with feeder cells and IL-2. After five days, the expaneded NK cells are transduced with a CAR of choice. Two days later, the CAR NK cells are subjected to CRISPR/Cas9 RNP to knockout one or more genes of interest, such as inhibitory genes. In specific embodiments, there is large-scale KO of one or more inhibitory genes, suppressive genes, checkpoint molecules, and so forth. Following the gene editing step, the modified cells are expanded with feeder cells and IL-2. Finally, the cells may be analyzed, such as for KO efficiency, using functional assays, cytotoxicity assays, in vivo activity, a combination thereof, and so forth.

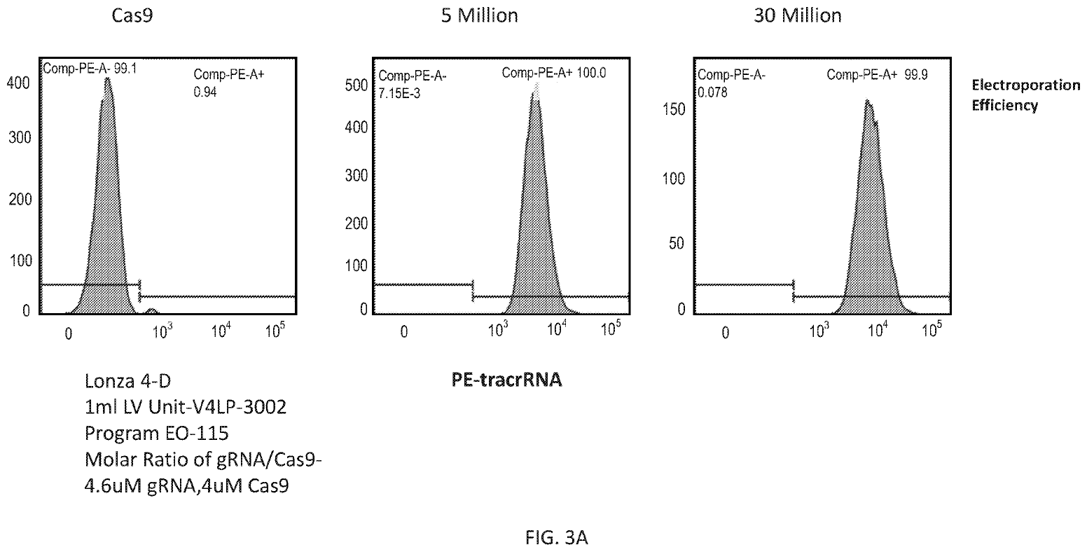

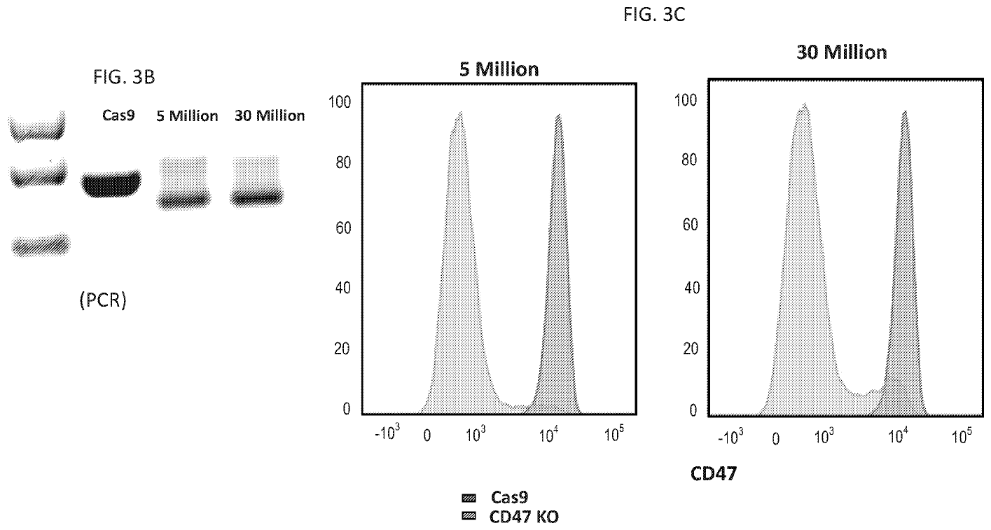

Small-scale and large-scale efficiency of CRISPR RNP-mediated KO of CD47 in primary CAR-NK cells was compared (FIG. 3). The efficiency of electroporation was demonstrated using a tracrRNA fluorescent label for 5 million and 30 million scale levels (FIG. 3A). In FIG. 3B, knockout efficiency was demonstrated using PCR gel electrophoresis at 5 million and 30 million electroporated cell dose levels. As demonstrated in FIG. 3C, knockout efficiency demonstrated using flow cytometry was similar at the small-scale and large-scale levels.

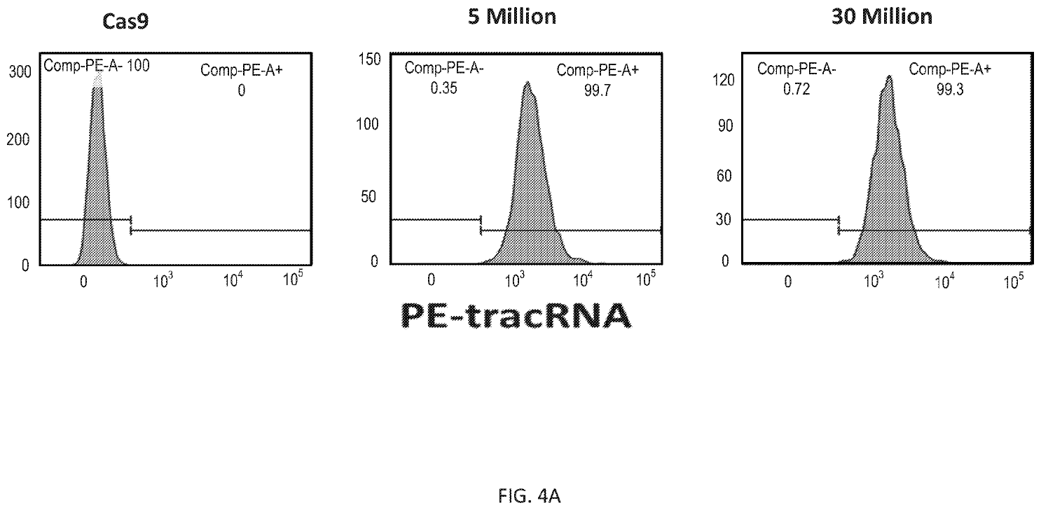

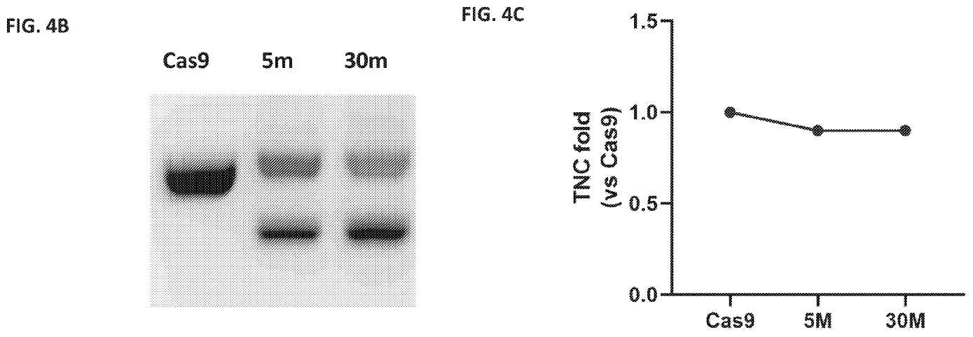

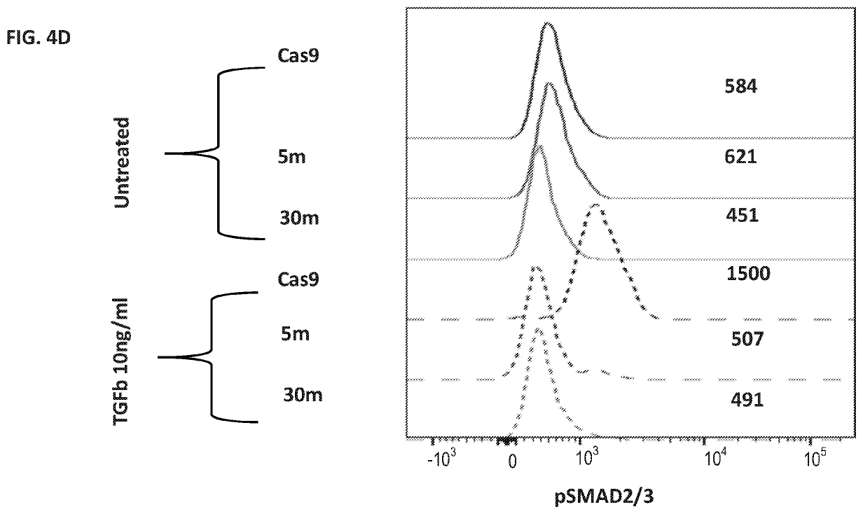

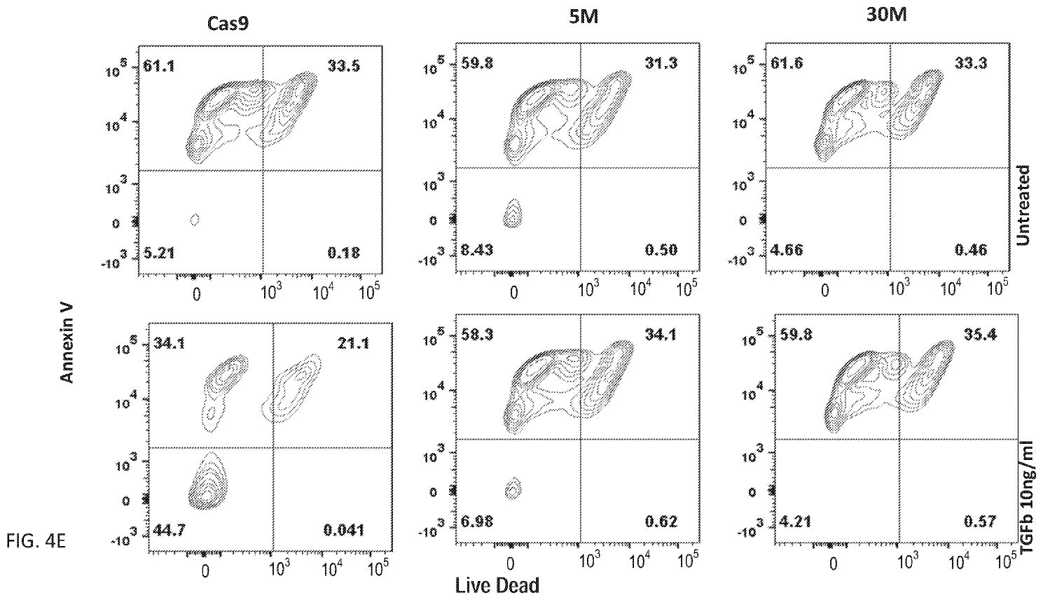

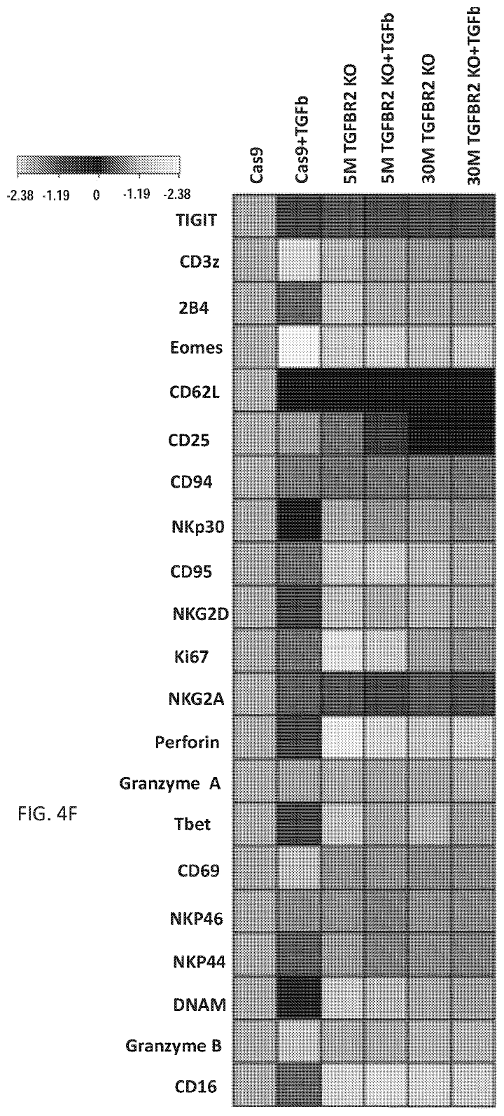

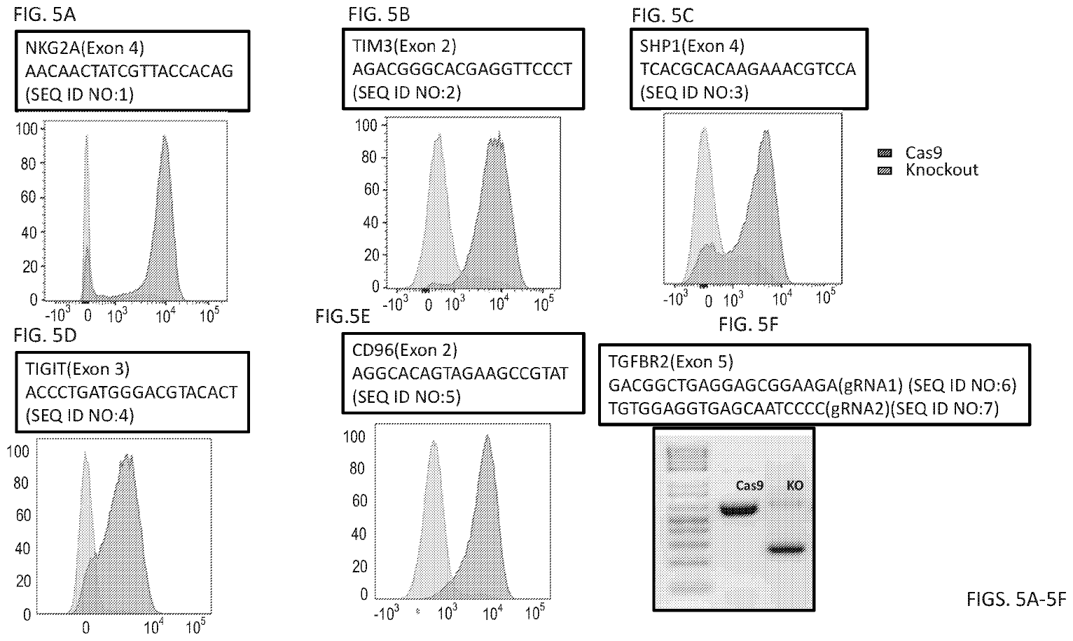

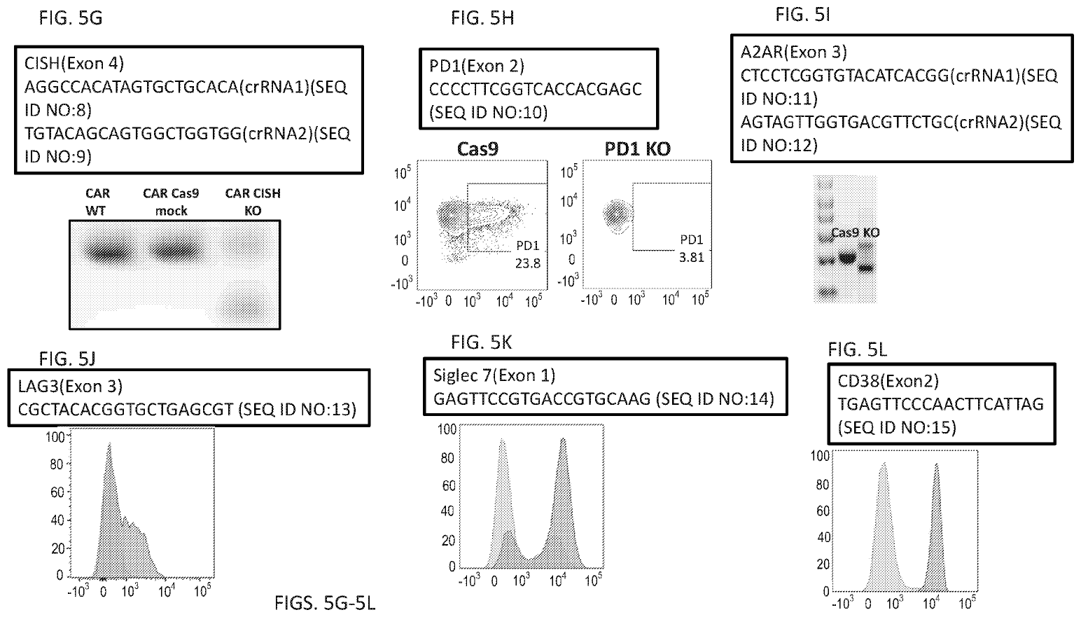

FIG. 4 concerns TGFBR2 KO at small-scale and large-scale using in CAR-NK cells. In particular, FIG. 4 provides another comparison of small-scale and large-scale CRISPR RNP-mediated KO of a different gene, TGFBR2, in primary CAR-NK cells. Electroporation efficiency, demonstrated by tracrRNA fluorescent label at 5 million and 30 million scale levels (FIG. 4A), and knockout efficiency, shown by PCR gel electrophoresis in 5 million vs. 30 million cells (FIG. 4B), are provided. In FIG. 4C, total nucleated live cell counts showed no difference in live cell numbers compared to Cas9 control following RNP-mediated KO at 5 million vs. 30 million cells. This demonstrates that cell viability is not affected. Phosphoflow assays assessed pSMAD2/3 signaling (FIG. 4D) with or without treatment with TGF-beta, and the data confirm efficient abrogation of downstream TGF-beta signaling following TGFBR2 KO at both the 5 million and 30 million dose levels. An apoptosis Annexin V assay gated on THP-1 tumor targets was performed (FIG. 5E) demonstrating that after 4hrs following co-culture with THP-1, Cas9 control CAR-NK cells treated with TGFB kill tumor targets less efficiently compared with TGFBR2 KO CAR-NK cells at both the 5 million and 30 million cell KO scales. Finally, FIG. 4F shows a heatmap summarizing phenotyping using mass cytometry for Cas9 control or TGFBR2 KO CAR NK cells at the 5 million and 30 million cell KO scale levels. This shows that TGFBR2 KO CAR NK cells are protected from the immunosuppressive phenotype changes induced by TGF-beta exposure, including downregulation of activation and cytotoxicity markers (e.g., NKP30, NKG2D, DNAM, perforin, and/or granzyme).

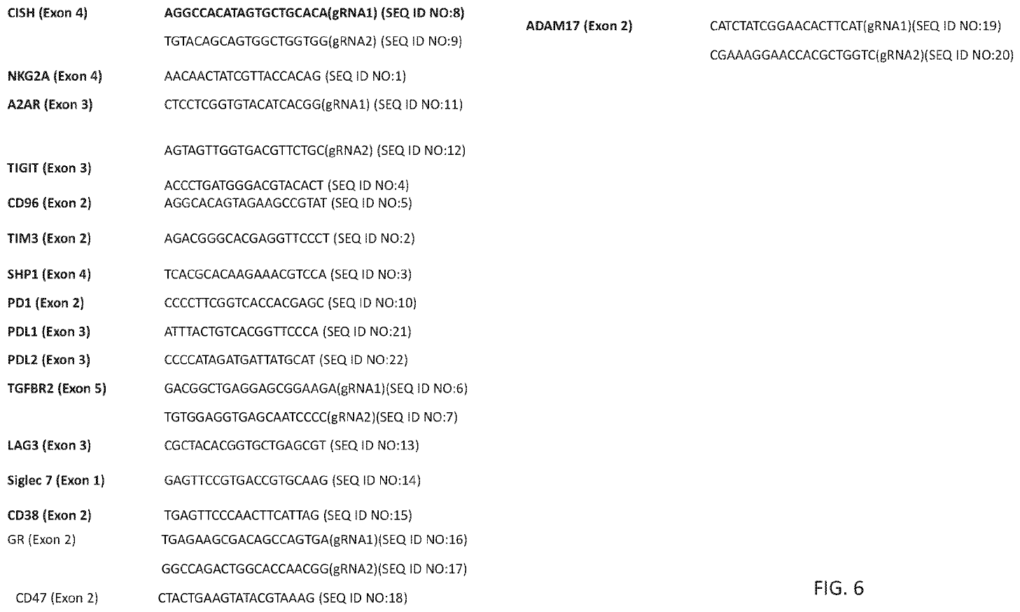

Examples of genes that may be knocked out and examples of their respective guide or crRNAs are provided in FIGS. 5A-5L, including corresponding exmples of KO efficiency as demonstrated by either flow cytometry or PCR. Other examples of sequences for targeting are provided in FIG. 6.

### Example 2

### Specific Examples of Large-Scale Combined CAR Transduction and CRISPR Gene Editing of NK Cells for Translation to the Clinic

The procedure for generating genetically modified natural killer (NK) cells using a viral vector and gene editing using Crispr Cas9 is as follows:

On Day 0, mononuclear cells are isolated from a single cord blood (CB) unit or from peripheral blood, washed and the CD3, CD14 and CD19 positive cells depleted using the CliniMACS immunomagnetic beads (Miltenyi Biotec). The unlabeled, enriched NK cells are collected, washed with CliniMACS buffer, counted, and combined with irradiated (100 Gy) APCs in a 1:2 ratio. The cell mixture (1×10⁶ cells/ml) is transferred to cell culture flasks containing NK Cell Complete Medium (NKCCM) (90% Stem Cell Growth Medium, 10% FBS, 2 mM L-glutamine) and IL-2, 200 U/mL. The cells are incubated at 37° C. in 5% CO₂. On Day 3, a media change is performed by collecting the cells by centrifugation and re-suspending them in NKCCM (1×10⁶ cells/ml) containing 200 U/mL of IL-2. The cells are then incubated at 37° C. in 5% CO₂. On Day 5, the number of wells needed for retronectin transduction is calculated based on the number of NK cells in culture. The retronectin solution is plated in 24-well culture plates. The plates are sealed and stored in a 4° C. refrigerator overnight or for 4-6 hours in 37° . On Days 5-6, a second NK cell selection is performed as described on Day 0 prior to transduction of the NK cells. The cells are washed with CliniMACS buffer, centrifuged and re-suspended in NKCCM at 0.5×10⁶/ml with IL-2, 600 U/ml. The Retronectin plates are then washed with NKCCM and incubated at 37° C. until use. The NKCCM in each well is then replaced with retroviral supernatant, followed by centrifugation of plates at 32° C. The retroviral supernatant is then aspirated and replaced with fresh retroviral supernatant. The NK cell suspension containing 0.5×10⁶ cells and 600 U/mL IL-2 is added to each well, and the plates centrifuged. The plates are then incubated at 37° C. with 5% CO₂. On day 7 or day 8, after checking the transduction efficiency (2 days after transduction with chimeric antigen receptor), cells are electroporated with a ribonucleoprotein (RNP) complex (see details of CRISPR cas9 gene editing for small-scale and large-scale expansion below) and the cells are cultured with uAPC cells at 1:2 ratio for an additional seven days. This approach can be taken to target either one or two genes simultaneously for deletion. If planning to target 3 or 4 genes simultaneously, first target two genes as described above. Then rest cells for 2-3 days in NKCM and then perform a second electroporation targeting the additional genes. On day 14 or 16 (if there are more than 2 genes) check knockout efficiency by flow cytometry, PCR, or western blotting.

The cells are ready for infusion on Days 15-17, in specific cases.

The following provides one particular example of a specific process for large-scale production of engineered NK cells:

1. CRISPR CAS9: Large-Scale Protocol (Starting NK Cell Number 5 -100 Million) crRNA Pre-Complexing and Electroporation (Lonza 4D)

Step 1: Make crRNA+tracrRNA duplex

| | | concen- | | | concen- |
|---|---|---|---|---|---|
| | volume | tration | | volume | tration |
| crRNA # 1 | 5 | 200 uM | crRNA # 2 | 5 | 200 uM |
| tracrRNA | 5 | 200 uM | tracrRNA | 5 | 200 uM |
| IDTE Buffer | 0 | | IDTE Buffer | 0 | |
| total volume | 10 ul | 100 uM | total volume | 10 ul | 100 uM |

The starting concentration of crRNA and tracrRNA are 200 uM. The final concentration after mixing them in equimolar concentration is 100 uM.

a. Mix with pipette, and centrifuge.
b. Incubate at 95° C. for 5 min in thermocycler.
c. Allow to cool to room temperature on the benchtop

Step 2: Combine the crRNA: tracrRNA duplex and Cas9 Nuclease

| | volume | volume |
|---|---|---|
| crRNA # 1: tracrRNA duplex | 1.2 ul(120 pmol) | 2.4 ul |
| (Step 1) | | |
| Cas9 (Undiluted) | 1.7 ul(104 pmol) | 3.4 ul |
| P3 Buffer | 2.1 | 4.2 |
| total volume | 5 ul | 10 ul |

| | volume |
|---|---|
| crRNA # 1, 2 + tracrRNA + cas9 (Step 3) | 20 ul |
| Cell Suspension | 100 ul |
| Total volume | 120 ul |

| | volume | volume |
|---|---|---|
| crRNA # 2: tracrRNA duplex | 1.2 ul(120 pmol) | 2.4 ul |
| (Step 1) | | |
| Cas9 (Step 2) | 1.7 ul(104 pmol) | 3.4 ul |
| P3 Buffer | 2.1 | 4.2 |
| total volume | 5 ul | 10 ul |

a. Mix with pipette, and centrifuge.
b. Incubate the mixture at room temperature for 15 min.

Step 3: Combine crRNA #1 and crRNA #2 from Step 3

| | volume | volume |
|---|---|---|
| crRNA # 1 + tracrRNA + cas9 (Step 3) | 5 ul | 10 ul |
| crRNA # 2 + tracrRNA + cas9 (Step 3) | 5 ul | 10 ul |
| total volume | 10 ul | 20 ul |

Step 4: Perform electroporation
a. Prepare culture plate with media (preferentially antibiotic free), with IL-2
b. Prepare 5E+106 cells (wash twice with PBS to remove FBS) and re-suspend in 100 ul P3 primary cell Nucleofector Solution just before use.
c. Mix Cell suspension and RNP(final Cas9 concentration is 4.6 uM, gRNA concentration is 4 uM), transfer to nucluocuvette and click lid into place.
d. The electroporation program is EO-115; the cells are then added to the culture plate and allowed to recover in 37 C.° incubator.
e. Up to 5E+106-X unit(Cat:AAF-1002X) (20 ul from last product is enough, see above for an example of final concentrations)
f. To electroporate up to 30×10⁶, use the 1 ml LV Kit L Unit (Cat. #: V4LC-2002). Calculate the total amount of RNP complex required by dividing the number of total cells by 5×10⁶ and then multiplying by the final amount of RNP complex from Step 3.
g. Example: if the cell count is 30×10⁶, 30×10⁶/5×10⁶=6, then 6×20 ul=X

The following provides a specific process for small-scale production of engineered NK cells.

CRISPR CAS9: Small-Scale Protocol (Starting Cell Population 0.25-3 million)

2. sgRNA-Cas9 Pre-Complexing and Electroporation(Neon-Thermo Fisher)
a. 1 or 2 sgRNAs spanning close regions were designed and used for each gene. 1.5 ug cas9 (PNA Bio) and 500 ng sgRNA (sum of all sgRNAs) reactions were made for each gene and incubated on ice for 20 minutes.
b. After 20 minutes, add 250,000 NK Cells re-suspended in T-buffer* (included with Neon Electroporation Kit, Invitrogen, total volume including RNP complex and cells should be 14u1) and electroporated with lOul electroporation tip using Neon Transfection System.
c. The electroporation conditions are 1600V, 10 ms, and 3 pulses for NK cells. The cells are then added to culture plate with APCs (1 NK: 2 APCs), SCGM media (preferentially antibiotic free) , 200 IU/ml IL2 and allowed to recover in 37 C incubator.
   3. crRNA Pre-Complexing and Electroporation (Neon-Thermo Fisher)

Step 1: Make crRNA+tracrRNA duplex

| | | concen- | | | concen- |
|---|---|---|---|---|---|
| | volume | tration | | volume | tration |
| crRNA # 1 | 2.2 ul | 200 uM | crRNA # 2 | 2.2 ul | 200 uM |
| tracrRNA | 2.2 ul | 200 uM | tracrRNA | 2.2 ul | 200 uM |
| IDTE Buffer | 5.6 ul | | IDTE Buffer | 5.6 ul | |
| total volume | 10 ul | 44 uM | total volume | 10 ul | 44 uM |

The starting concentration of crRNA and tracrRNA are 200 uM. The final
concentration after mixing them in equimolar concentration is 44 uM.

d. Mix with pipette, and centrifuge.
e. Incubate at 95 C for 5 min in thermocycler.
f. Allow to cool to room temperature on the benchtop.

Step 2: Prepare cas9 Nuclease

| | volume |
|---|---|
| Alt-R S.p. Cas9 Nuclease | 3 ul |
| 3NLS (61 uM) | |
| T buffer | 7 ul |
| total volume | 10 ul |
| final concentration | 18 uM |

Step 3: Combine the crRNA: tracrRNA duplex and Cas9 Nuclease

| | volume |
|---|---|
| crRNA # 1: tracrRNA duplex | 2 ul |
| (Step 1) | |
| Cas9 (Step 2) | 2 ul |
| total volume | 4 ul |

| | volume |
|---|---|
| crRNA #2: tracrRNA | 2 ul |
| duplex (Step 1) | |
| Cas9 (Step 2) | 2 ul |
| Total volume | 4 ul |

g. Mix with pipette, and centrifuge.
h. Incubate the mixture at room temperature for 15 min.

Step 4: Combine crRNA #1 and crRNA #2 from Step 3

| | volume |
|---|---|
| crRNA # 1 + tracrRNA + cas9 (Step 3) | 2.25 ul |
| crRNA # 2 + tracrRNA + cas9 (Step 3) | 2.25 ul |
| total volume | 4.5 ul |

| | |
|---|---|
| crRNA # 1, 2 + tracrRNA + cas9 (Step 3) | 4.5 ul |
| Cell Suspension | 7.5 ul |
| total volume | 12 ul |

Step 5: Perform electroporation
Prepare 250,000 cells per well and re-suspend in 7.5 ul T buffer just before use.
The electroporation conditions are 1600V, 10 ms, and 3 pulses. The cells are then added to culture plate and allowed to recover in 37 C incubator.

### Example 3

### Multiplex Gene Editing in NK Cells and CAR-NK Cells

The present examples concerns production of NK cells that both express one or more engineered antigen receptors, such as a CAR, and also have been gene edited to reduce or eliminate expression of one or more endogenous genes. In FIG. 2, there is one example of a method of combining CAR transduction with multiplex gene editing in NK cells. NK cells are obtained from a suitable source, such as cord blood, and expanded in a suitable manner, such as with feeder cells and one or more suitable cytokiens, such as IL-2, for example. The expanded cells in specific cases are first subjected to modification to express a CAR, in a specific example. The step of transduction of the NK cells may occur in a particular amount of time of expansion, such as at day 5 or about day 5 since the beginning of the expansion step. The NK cells may be transduced with standard methods of transduction for NK cells to produce CAR-NK cells. Within a suitable amount of time, such as at day 7 (or about day 7) since the beginning of the expansion step, the CAR-NK cells ares subject to gene editing modification, and in specific cases this is performed as a knockout using CRISPR/Cas9 RNP. In specific cases, the gene editing step is large scale knockout of one or more endogenous genes in the CAR-NK cells. Certain examples of genes that are knocked out include TGF-beta, TIGIT, and/or ADAM17. In some embodiments, the produced CAR-NK KO cells are expanded under suitable conditions, such as with appropriate feeder cells and one or more cytokines, such as IL-2, for example. Following a suitable duration of time, the CAR-NK KO cells are first assessed for one or more activities prior to use, although in other instances they are not assessed for the one or more activities. In some cases, the CAR-NK KO cells are assessed for KO efficiency, functional assays, cytotoxicity assays, and/or in-vivo activity, as examples only.

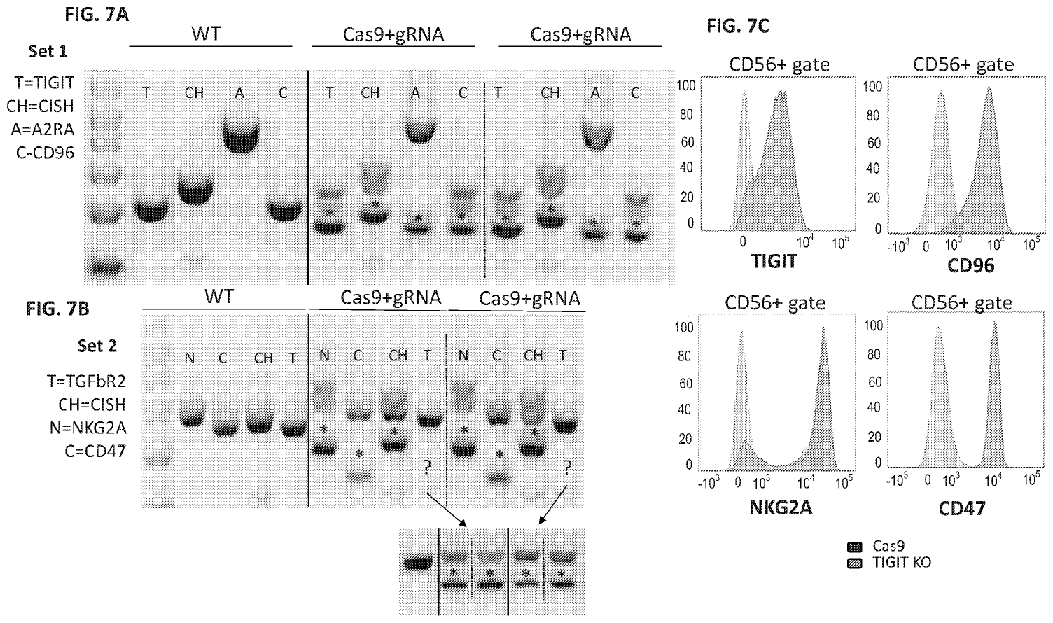

Following production of the CAR-NK KO cells, the produced cells may be assessed. As one example, they may be assessed for the efficiency of gene editing with respect to disruption of the desired endogenous genes in the NK cells. FIG. 7 illustrates one example where the CAR-NK cells were gene disrupted for the noted genes and assessed with PCR. In FIGS. 7A and 7B, the PCR results show that CRISPR/Cas9 mediates efficient multiple genes disruption in NK cells. Another means for demonstrating efficiency of KO is by flow cytometry, as demonstrated in FIG. 7C.

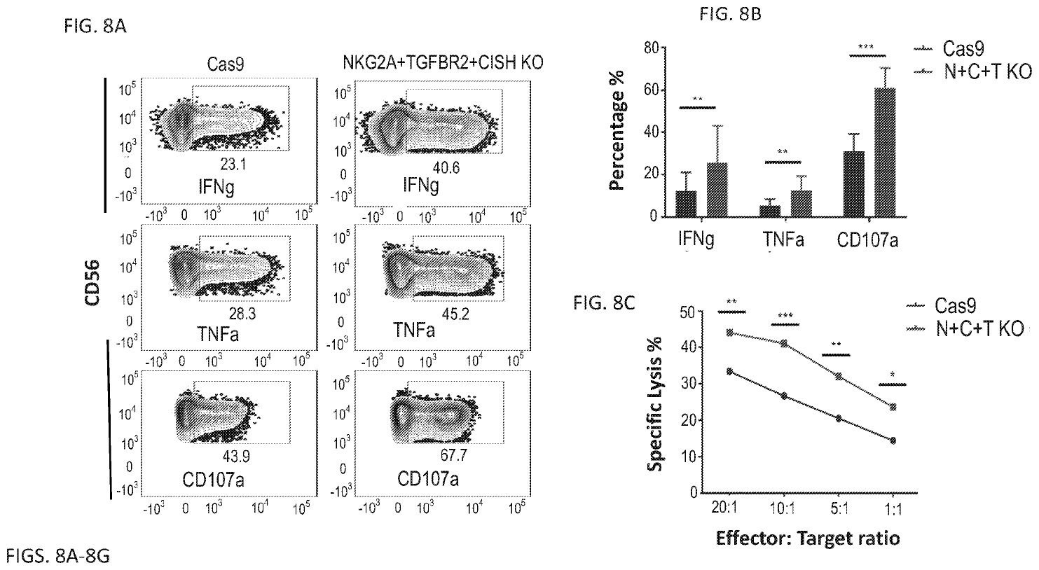

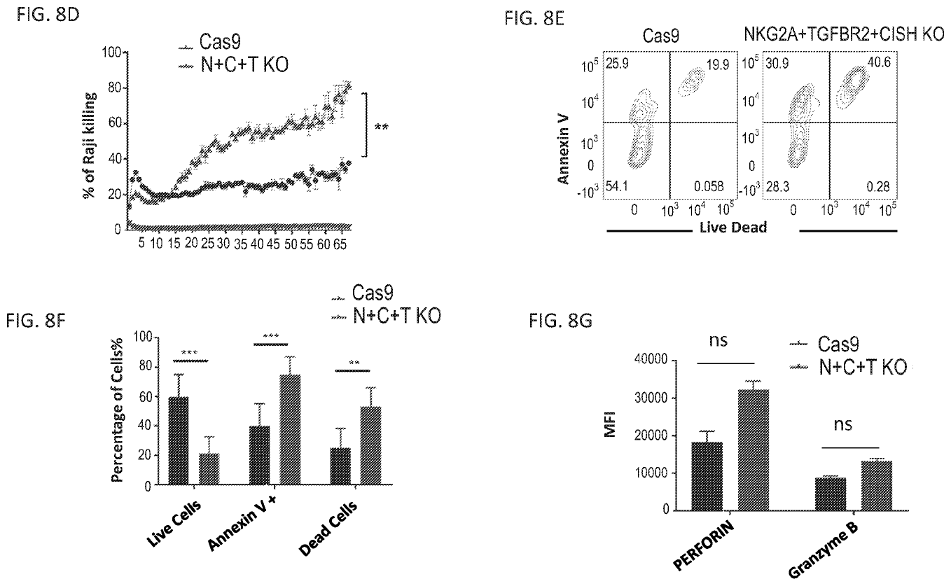

Following production of the CAR-NK KO cells, one can assess their in vitro or in vivo activity. FIGS. 8A-8H show that multiplex gene editing of inhibitory molecules in NK cells improves their in vitro function and anti-tumor activity. Specifically, FIG. 8 concerns NK cells that express a CD19 CAR and that were knocked out for three genes: NKG2A, TGFBR2, and CISH. FIG. 8A The cells were functionally assessed for particular cytokine production (here, IFNg and TNFa) in addition to the status of degranulation (as determined by CD107a). The FACS plots in FIG. 8A show both an increased IFNg and TNFa production and degranulation as compared to control (see also FIG. 8C). In FIG. 8C, cytotoxicity was assessed by chromium release assay, here showing increased specific lysis of Raji tumor cells by the triple KO CAR19-NK cells compared to a Cas9 control. Another cytotoxicity assay, Incucyte killing assay, confirms enhanced killing of Raji tumor cells by the triple KO CAR19-NK cells (FIG. 8D). The triple KO CAR19-NK cells also demonstrated increased apotosis of Raji tumor cells, as demonstrated by FACS plot of an Annexin V apoptosis assay (FIG. 8E, 8F). Finally, FIG. 8G demonstrates NK-cell cytotoxicity by showing increased Perforin and Granzyme B production by the triple KO CAR19-NK cells.

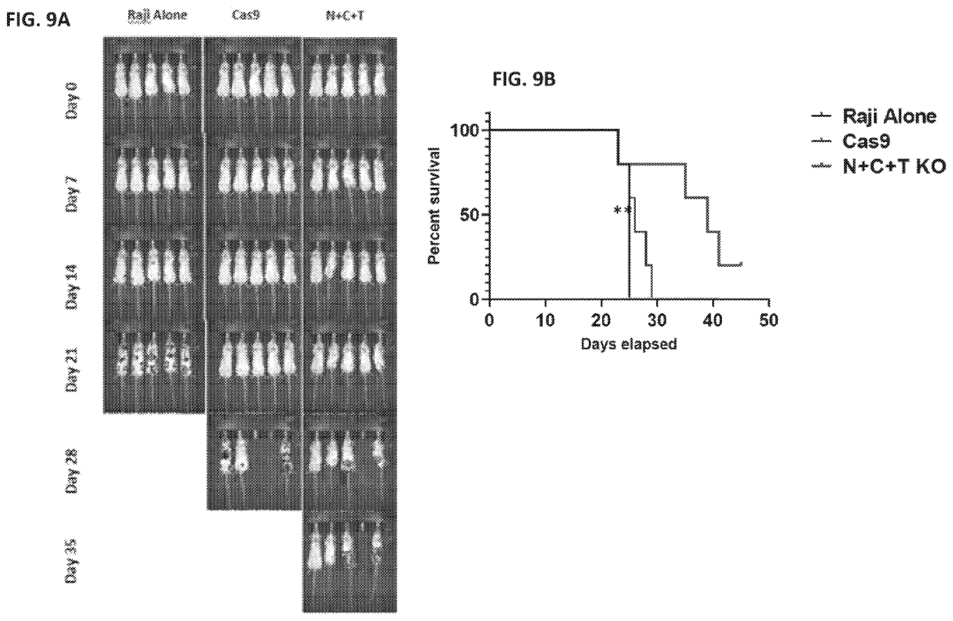

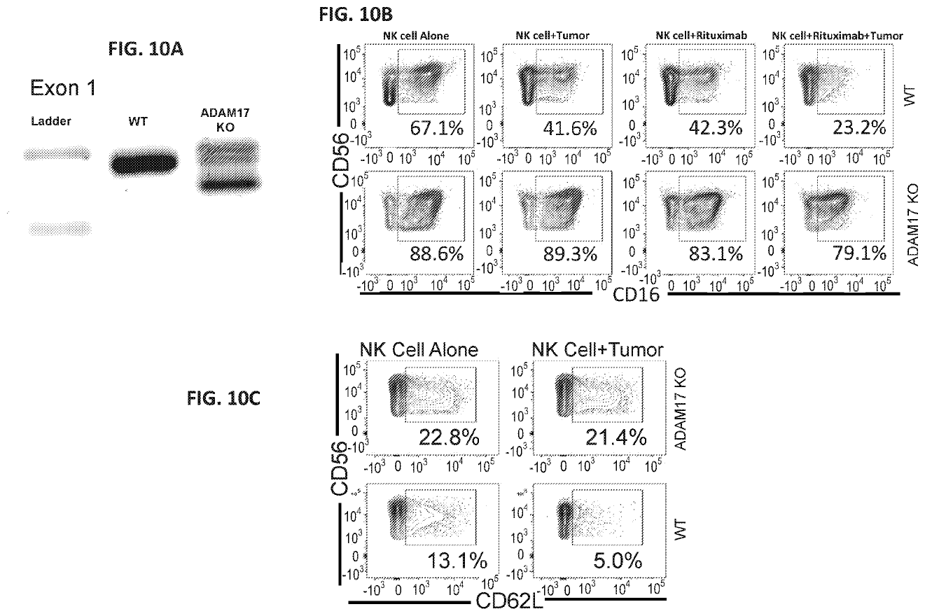

The cells may be assessed for in vivo activity. In FIG. 9A-9B, the cells were assessed for anti-tumor activity using BLI imaging. There was improved tumor control in the triple KO CAR19-NK cells as compared to control, indicating that multiplex gene editing of inhibitory molecules in NK cells improves their in vivo anti-tumor efficacy and also enhances survivial (FIG. 10B).

In one example, the CAR NK cells may be knocked out for an endogenous gene that prevents shedding of CD16 and CD62L (prevention of shedding of CD16 can preserve antibody cellular cytotoxicity, and prevention of shedding of CD62L can improve homing to bone marrow and lymph nodes). In specific cases, the CAR NK cells are knocked out for ADAM17, and FIGS. 10A-10C demonstrate that ADAM17 KO in CAR-NK cells prevents shedding of CD16 and CD62L.

### Example 4

### Large Scale NR3C1 Knockout Protocol

This present aspect concerns one example of a large-scale knockout protocol for the gene NR3C1. In this particular case, as an example, the gene editing was performed by CRISPR techniques.

Examples of Materials
1. gRNA #1 (61 uM, Synthego)
2. gRNA #2 (61 uM, Synthego)
3. SpyFY Cas9 (61 uM, Aldevron)
4. Plasmalyte supplemented with HEPES (15 mM) and Mannitol (50 mM)
5. Lonza 4D Nucleofactor (Program: Primary P3, CA137, CM137)
6. Culture flasks

Procedure: Below is a protocol for 100E6 cells

Step 1: Combine gRNA and cas9 together in the molar ratio of 3:1. Do this separately for all gRNAs.

| | |
|---|---|
| volume | concentration |

| | | | |
|---|---|---|---|
| gRNA # 1 | 22.50 | ul | 36.6 uM |
| Cas9 | 7.5 | ul | 12.2 uM |
| Plasmalyte | 7.5 | ul | |
| total volume | 5 | ul | |

| | |
|---|---|
| volume | concentration |

| | | | |
|---|---|---|---|
| gRNA # 2 | 22.5 | ul | 36.6 uM |
| Cas9 | 7.5 | ul | 12.2 uM |
| Plasmalyte | 7.5 | ul | |
| total volume | 5 | ul | |

Mix with pipette, and centrifuge.

Incubate the mixture at room temperature for 20 min.

Step 2: Combine the gRNA #1+ Cas9 and gRNA#2+ Cas9 in 1:1 ratio

| | volume | concentration |
|---|---|---|
| gRNA1 + Cas9 | 37.5 ul | 18.8 uM/6.1 uM |
| gRNA2 + Cas9 | 37.5 ul | 18.8 uM/6.1 uM |
| total volume | 10 ul | |

Step 3: Perform electroporation of cells.

Prepare culture flasks with appropriate media prior to electroporation.

Prepare 100E6 cells and re-suspend in 1 ml Plasmalyte just prior to use.

| | volume | concentration |
|---|---|---|
| gRNA #1, 2 + Cas9 | 75 ul | 1 uM/300 ng |
| Cell suspension | 1000 ul | |
| total volume | 1075 ul | |

Use Lonza 4D Nucleofactor system to electroporate using program code CA137 or CM137

Electroporate and add to pre-prepared flasks.

Mix and incubate in 37° C.

After 3 days, check knockout efficiency by PCR and/or western blot, for example.

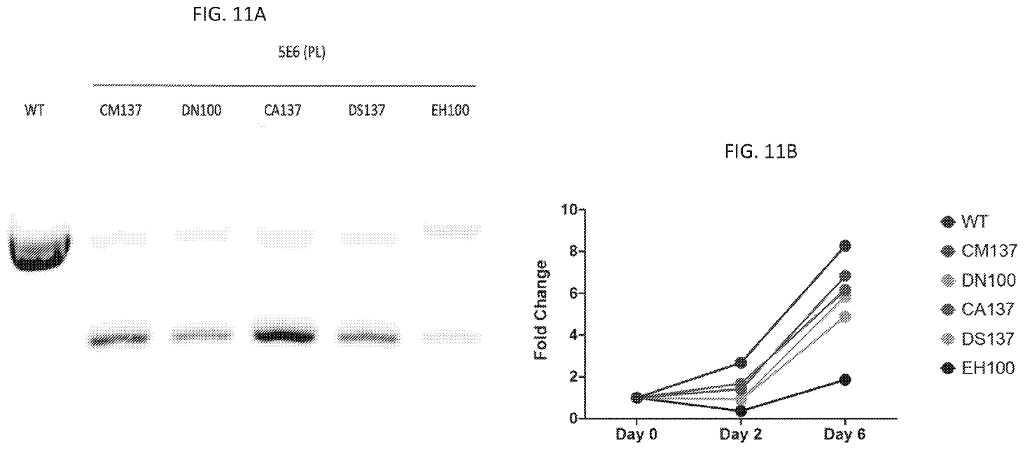

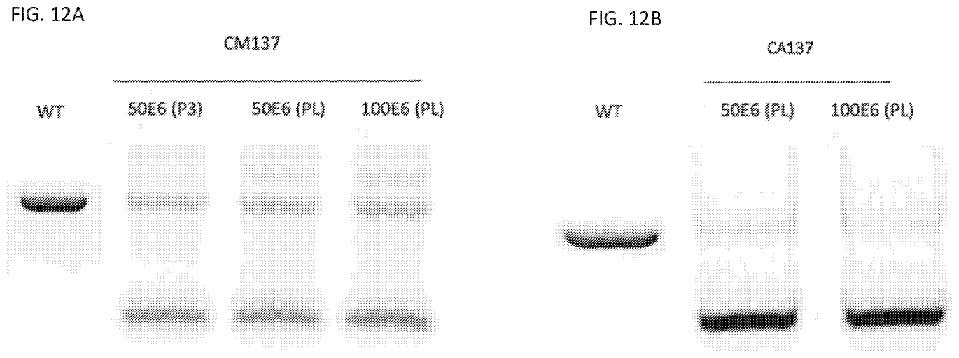

FIG. 11A demonstrates NR3C1 knockout efficiency being tested using PCR for various programs (CM137, DN100, CA137, DS137 and EH100) used for electroporation in Lonza 4D-Nucleofactor™. This was done in small scale, with 5E6 T cells and Plasmalyte (supplemented with HEPES and mannitol, plasmalyte (PL)). Knockout efficiency using CA137 and CM137 was higher compared to other programs from the manufacturer. The fold expansion of the NR3C1 knocked out cells was tested with the varous electroporation programs from the manufacturer (FIG. 11B). Cell viability and expansion rate were higher with CA137 and CM137. Large scale studies (FIGS. 12A-12B) were then performed to test feasibility of these two programs.

In FIG. 12A, NR3C1 knockout efficiency using PCR was tested for the CM137 program used for electroporation in Lonza 4D-Nucleofactor™. This was done in large scale, with 50E6 T cells, using either Lonza buffer (P3) or PL (supplemented with HEPES and mannitol) and with 100E6 T cells using PL (supplemented with HEPES and mannitol). Knockout efficiency and viability was comparable for all conditions. FIG. 12B demonstrates NR3C1 knockout efficiency using the CA137 program utilized for electroporation in Lonza 4D-Nucleofactor™. This was done in large scale, with either 50E6 T cells or 100E6 T cells using PL (supplemented with HEPES and mannitol). Knockout efficiency and viability was comparable for all conditions.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ausubel et al., Current Protocols in Molecular Biology, Greene Publishing Associates and John Wiley & Sons, NY, 1994.
Bennekov et al., Mt. Sinai J. Med. 71 (2): 86-93, 2004.
Chothia et al., EMBO J. 7:3745, 1988.
Cohen et al. J Immunol. 175:5799-5808, 2005.
Davila et al. PLoS ONE 8(4): e61338, 2013.
European patent application number EP2537416
Fedorov et al., Sci. Transl. Medicine, 5(215), 2013.
Frolet et al., BMC Microbiol. 10:190 (2010).
Hanibuchi et al., Int. J. Cancer, 78(4):480-485, 1998.
Heemskerk et al. Hum Gene Ther. 19:496-510, 2008.
International Patent Publication No. WO 00/37504
International Patent Publication No. WO 01/14424
International Patent Publication No. WO 14/055668
International Patent Publication No. WO 00/14257
International Patent Publication No. WO 01/014424
International Patent Publication No. WO 07/103009
International Patent Publication No. WO 12/129514
International Patent Publication No. WO 13/071154
International Patent Publication No. WO 13/123061
International Patent Publication No. WO 13/166321
International Patent Publication No. WO 13/126726
International Patent Publication No. WO 14/031687
International Patent Publication No. WO 99/40188
Janeway et al, Immunobiology: The Immune System in Health and Disease, 3^{rd} Ed., Current Biology Publications, p. 433, 1997.
Johnson et al. Blood 114:535-46, 2009.
Jores et al., PNAS U.S.A. 87:9138, 1990.
Lefranc et al., Dev. Comp. Immunol. 27:55, 2003.
Linnemann, C. et al. Nat Med 21, 81-85, 2015.
Lockey et al., Front. Biosci. 13:5916-27, 2008.
Loewendorf et al., J. Intern. Med. 267(5):483-501, 2010.
Marschall et al., Future Microbiol. 4:731-42, 2009.
Parkhurst et al. Clin Cancer Res. 15: 169-180, 2009.
Rieder et al., J. Interferon Cytokine Res. (9):499-509, 2009.
Rykman, et al., J. Virol. 80(2):710-22, 2006.
Sadelain et al., Cancer Discov. 3(4): 388-398, 2013.
Sambrook et al., Molecular Cloning: A Laboratory Manual, 3^{rd} ed., Cold Spring Harbor Press, Cold Spring Harbor, N.Y. 2001.
Shah et al., PLoS One, 8:e776781, 2013.
Singh et al., Cancer Research, 68:2961-2971, 2008.
Singh et al., Cancer Research, 71:3516-3527, 2011.
Turtle et al., Curr. Opin. Immunol., 24(5): 633-39, 2012.
U.S. Pat. No. 4,870,287
U.S. Pat. No. 5,760,395
U.S. Pat. No. 5,801,005
U.S. Pat. No. 5,994,136
U.S. Pat. No. 6,013,516
U.S. Pat. No. 6,225,042
U.S. Pat. No. 6,355,479
U.S. Pat. No. 6,410,319
U.S. Pat. No. 6,416,998
U.S. Pat. No. 6,544,518
U.S. Pat. No. 7,109,304
U.S. Pat. No. 7,446,190
U.S. Pat. No. 7,989,425
U.S. Pat. No. 8,017,114
U.S. Pat. No. 8,071,369
U.S. Pat. No. 8,354,509
U.S. Patent Publication No. 2002131960
U.S. Patent Publication No. 2005/0260186
U.S. Patent Publication No. 2006/0104968
U.S. Patent Publication No. 2009/0004142
U.S. Patent Publication No. 2009/0017000
U.S. Patent Publication No. 20130149337
U.S. Patent Publication No. 2013287748
Varela-Rohena et al. Nat Med. 14: 1390-1395, 2008.
Wu et al., Adv. Cancer Res., 90: 127-56, 2003.
Wu et al., Cancer, 18(2): 160-75, 2012.
Yu et al., Science, 318:1917-1920, 2007.
Zysk et al., Infect. Immun. 68(6):3740-43, 2000.

What is claimed is:

1. An in vitro method of producing engineered NK cells, comprising the steps of:
(a) expanding for a first time period NK cells with antigen presenting cells and an effective amount of interleukin (IL)-2 to produce expanded NK cells;
one of (b1) and (c1), or (b2) and (c2):
(b1) transducing or transfecting the expanded NK cells of (a) with one or more vectors each encoding one or more heterologous antigen receptors to produce modified NK cells;
(c1) after a second time period, delivering to the modified NK cells an effective amount of Cas9 and one or more guide RNAs to disrupt expression of one or more genes in the NK cells, thereby producing gene edited modified NK cells; or
(b2) delivering to the expanded NK cells of (a) an effective amount of Cas9 and one or more guide RNAs to disrupt expression of one or more genes in the NK cells, thereby producing gene edited NK cells;
(c2) after a second time period, transducing or transfecting the gene edited NK cells with one or more vectors each encoding one or more heterologous antigen receptors to produce gene edited modified NK cells; and
(d) expanding for a third time period the gene edited modified NK cells of (c1) or (c2) in the presence of an effective amount of antigen presenting cells and an effective amount of IL-2.

2. The method of claim 1, wherein the gene edited modified NK cells are produced from the steps of (b1) and (c1).

3. The method of claim 1, wherein the gene edited modified NK cells are produced from the steps of (b2) and (c2).

4. The method of claim 1, wherein the steps of (c1) and (b2) are each further defined as two or more delivering steps.

5. The method of claim 4, wherein a first delivering step comprises delivering guide RNAs that target one or more genes and a second delivering step comprises delivering guide RNAs that target one or more genes that are different from the one or more genes in the first delivering step.

6. The method of claim 5, wherein the duration between the first and second delivering steps is at least about two days.

7. The method of claim 5, wherein the duration between the first and second delivering steps is about two to three days.

8. The method of any one of claims 1-7, wherein the first time period is between about 5-7 days.

9. The method of any one of claims 1-8, wherein the second time period is about 2 days.

10. The method of any one of claims 1-9, wherein the third time period is at least about 7 days.

11. The method of any one of claims 1-10, wherein the third time period is between about 1-2 weeks.

12. The method of any one of claims 1-11, wherein a delivering step is by electroporation.

13. The method of claim 12, wherein an electroporation uses between about 200,000 cells to 1×10⁹ NK cells.

14. The method of claim 12, wherein an electroporation uses between about 200,000 and 2,000,000 cells.

15. The method of claim 12, wherein an electroporation uses between about 1,000,000 to 1×10⁹ NK cells.

16. The method of any one of claims 1-15, further comprising a step of depleting NK cells of CD3-positive, CD14-positive, and/or CD19-positive cells prior to (a).

17. The method of any one of claims 1-16, wherein in (a) and/or (d) the ratio of NK cells to antigen presenting cells is 1:1, 1:1.5, 1:2, or 1:3.

18. The method of claim 12, wherein the concentration of the guide RNA in the electroporation step is 3, 4, or 5 μM.

19. The method of claim 12, wherein the concentration of the Cas9 nuclease in the electroporation step is 4.0, 4.1, 4.2, 4.3, 4.4, 4.5, 4.6, 4.7, 4.8, 4.9, or 5 μM.

20. The method of any one of claims 1-19, wherein the concentration of IL-2 is between about 100 units/ml to about 300 units/ml.

21. The method of claim 20, wherein the concentration of IL-2 is 100, 125, 150, 175, 200, 225, 250, 275, or 300 units/ml.

22. The method of any one of claims 1-21, further comprising the step of depleting NK cells of CD3-positive, CD14-positive, and/or CD19-positive cells prior to step (b1) or (b2).

23. The method of any one of claims 1-22, wherein the NK cells in (a) are derived from cord blood, peripheral blood, bone marrow, stem cells, cell lines, or a mixture thereof.

24. The method of any one of claims 1-23,wherein the heterologous antigen receptor is a chimeric antigen receptor or a T cell receptor.

25. The method of any one of claims 1-24, wherein the heterologous antigen receptor targets a tumor associated antigen.

26. The method of any one of claims 1-24, wherein the heterologous antigen receptor targets an antigen selected from the group consisting of CD19, EBNA, CD123, HER2, CA-125, TRAIL/DR4, CD20, CD70, HLA-G, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases, MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, GAGE -2, GAGE-3, GAGE-4, GAGE-5, GAGE-6, GAGE-7A, GAGE-7B, GAGE-8, NA88-A, MC1R, MDA-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, a Phosphoinositide 3-kinase, a TRK receptor, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1), TACSTD2, a receptor tyrosine kinase, Epidermal Growth Factor receptor (EGFR), EGFRvIII, platelet derived growth factor receptor (PDGFR), vascular endothelial growth factor receptor (VEGFR), VEGFR2, a cytoplasmic tyrosine kinase, integrin-linked kinase (ILK), signal transducers and activators of transcription STAT3, STATS, and STATE, HIF-1, HIF-2, Nuclear Factor-Kappa B (NF-B), a Notch receptor NY ESO 1, c-Met, mammalian targets of rapamycin (mTOR), WNT, extracellular signal-regulated kinases (ERKs), PMSA, PR-3, MDM2, Mesothelin, renal cell carcinoma-5T4, SM22-alpha, carbonic anhydrases I (CAI), CAIX), STEAD, TEL/AML1, GD2, proteinase3, hTERT, sarcoma translocation breakpoints, EphA2, ML-IAP, EpCAM, TMPRSS2 ETS fusion gene, ERG, NA17, PAX3, ALK, androgen receptor, cyclin B 1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAXS, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, and a combination thereof.

27. The method of any one of claims 1-26, wherein the gene that has disruption of expression in the NK cells is an inhibitory gene.

28. The method of claim 27, wherein the inhibitory gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, CD39, and a combination thereof.

29. The method of any one of claims 1-28, wherein following (d) the cells are analyzed.

30. The method of claim 29, wherein the cells are analyzed by one or more functional assays, one or more cytoxicity assays, and/or in vivo activity.

31. The method of claim 29 or 30, wherein the cells are analyzed by flow cytometry, mass cytometry, RNA sequencing, or a combination thereof.

32. The method of any one of claims 1-31, wherein following (d) the cells are stored.

33. The method of any one of claims 1-32, wherein following (d) the cells are cryopreserved.

34. The method of any one of claims 1-33, wherein following (d) an effective amount of the cells are delivered to an individual in need thereof.

35. The method of claim 34, wherein the individual has cancer, an infectious disease, or an immune-related disorder.

36. A population of NK cells produced by the method of any one of claims 1-35.

37. A composition comprising the population of claim 36.

38. The composition of claim 37, formulated in a pharmaceutically acceptable carrier.

39. A method of treating an individual for a medical condition, comprising the step of administering to the individual a therapeutically effective amount of NK cells produced by the method of any one of claims 1-35.

40. The method of claim 39, wherein the medical condition is cancer.

41. The method of claim 40, wherein the cancer comprises a hematological malignancy or a solid tumor.

42. The method of claim 39, wherein the medical condition is infectious disease and/or an immune-related disorder.

43. The method of any one of claims 39-42, wherein the NK cells are delivered to the individual once or multiple times.

44. The method of claim 43, wherein when the NK cells are delivered to the individual multiple times, the duration between administrations to the individual comprises 1, 2, 3, 4, 5, 6, 7, 8, 9, 10, 11, 12, 13, 14, 15, 16, 17, 18, 19, 20, 21, 22, 23, or 24 hours.

45. The method of claim 43, wherein when the NK cells are delivered to the individual multiple times, the duration between administrations to the individual comprises 1, 2, 3, 4, 5, 6, or 7 days.

46. The method of claim 43, wherein when the NK cells are delivered to the individual multiple times, the duration between administrations to the individual comprises 1, 2, 3, 4, 5, 6, 7, 8, 9, 10, 11, or 12 months.

47. The method of any one of claims 39-46, wherein the individual is administered an effective amount of one or more additional therapies for the medical condition.

48. The method of claim 47, wherein the additional therapy is administered to the individual prior to, during, and/or subsequent to the administration of the NK cells.

49. A kit comprising the NK cells produced by the method of any one of claims 1-35, and/or one or more reagents to produce the NK cells.
